SEQUENCE LISTING (6 sequences)

SEQ ID NO: 1 - DNA, 1717 bp, Homo sapiens
ctgccagggggagggccccagagaaaaccagaaagagggtgagagactgaggaagataaagcgtcccagggcctcctacaccagcgcctgagcaggaagcgggaggggccatgactacgaggccctgggaggtcactttagggagggctgtcctaaaaccagaagcttggagcagaaagtgaaaccctggtgctccagacaaagatcttagtcgggactagccggccaaggatgaagcctcacttcagaaacacagtggagcgaatgtatcgagacacattctcctacaacttttataatagacccatcctttctcgtcggaataccgtctggctgtgctacgaagtgaaaacaaagggtccctcaaggccccctttggacgcaaagatctttcgaggccaggtgtattccgaacttaagtaccacccagagatgagattcttccactggttcagcaagtggaggaagctgcatcgtgaccaggagtatgaggtcacctggtacatatcctggagcccctgcacaaagtgtacaagggatatggccacgttcctggccgaggacccgaaggttaccctgaccatcttcgttgcccgcctctactacttctgggacccagattaccaggaggcgcttcgcagcctgtgtcagaaaagagacggtccgcgtgccaccatgaagatcatgaattatgacgaatttcagcactgttggagcaagttcgtgtacagccaaagagagctatttgagccttggaataatctgcctaaatattatatattactgcacatcatgctgggggagattctcagacactcgatggatccacccacattcactttcaactttaacaatgaaccttgggtcagaggacggcatgagacttacctgtgttatgaggtggagcgcatgcacaatgacacctgggtcctgctgaaccagcgcaggggctttctatgcaaccaggctccacataaacacggtttccttgaaggccgccatgcagagctgtgcttcctggacgtgattcccttttggaagctggacctggaccaggactacagggttacctgcttcacctcctggagcccctgcttcagctgtgcccaggaaatggctaaattcatttcaaaaaacaaacacgtgagcctgtgcatcttcactgcccgcatctatgatgatcaaggaagatgtcaggaggggctgcgcaccctggccgaggctggggccaaaatttcaataatgacatacagtgaatttaagcactgctgggacacctttgtggaccaccagggatgtcccttccagccctgggatggactagatgagcacagccaagacctgagtgggaggctgcgggccattctccagaatcaggaaaactgaaggatgggcctcagtctctaaggaaggcagagacctgggttgagcctcagaataaaagatcttcttccaagaaatgcaaacaggctgttcaccaccatctccagctgatcacagacaccagcaaagcaatgcactcctgaccaagtagattcttttaaaaattagagtgcattactttgaatcaaaaatttatttatatttcaagaataaagtactaagattgtgctcaatacacagaaaagtttcaaacctactaatccagcgacaatttgaatcggttttgtaggtagaggaataaaatgaaatactaaatctttctgtaaaaaaaaaa

SEQ ID NO: 2 - protein, 384 aa, Homo sapiens
MKPHFRNTVERMYRDTFSYNFYNRPILSRRNTVWLCYEVKTKGPSRPPLDAKIFRGQVYSELKYHPEMRFFHWFSKWRKLHRDQEYEVTWYISWSPCTKCTRDMATFLAEDPKVTLTIFVARLYYFWDPDYQEALRSLCQKRDGPRATMKIMNYDEFQHCWSKFVYSQRELFEPWNNLPKYYILLHIMLGEILRHSMDPPTFTFNFNNEPWVRGRHETYLCYEVERMHNDTWVLLNQRRGFLCNQAPHKHGFLEGRHAELCFLDVIPFWKLDLDQDYRVTCFTSWSPCFSCAQEMAKFISKNKHVSLCIFTARIYDDQGRCQEGLRTLAEAGAKISIMTYSEFKHCWDTFVDHQGCPFQPWDGLDEHSQDLSGRLRAILQNQEN

SEQ ID NO: 3 - DNA, 4706 bp, Homo sapiens
ttccctttgcaattgccttgggtcctgccgcacagagcggcctgtctttatcagaggtccctctgccagggggagggccccagagaaaaccagaaagagggtgagagactgaggaagataaagcgtcccagggcctcctacaccagcgcctgagcaggaagggggaggggccatgactacgaggccctgggaggtcactttagggagggctgtcctgaaacctggagcctggagcagaaagtgaaaccctggtgctccagacaaagatcttagtcgggactagccggccaaggatgaagcctcacttcagaaacacagtggagcgaatgtatcgagacacattctcctacaacttttataatagacccatcctttctcgtcggaataccgtctggctgtgctacgaagtgaaaacaaagggtccctcaaggccccgtttggacgcaaagatctttcgaggccaggtgtattcccagcctgagcaccacgcagaaatgtgcttcctctcttggttctgtggcaaccagctgcctgcttacaagtgtttccagatcacctggtttgtatcctggaccccctgcccggactgtgtggcgaagctggccgaattcctggctgagcaccccaatgtcaccctgaccatctccgccgcccgcctctactactactgggaaagagattaccgaagggcgctctgcaggctgagtcaggcaggggcccgcgtgaagattatggacgatgaagaatttgcatactgctgggaaaactttgtgtacagtgaaggtcagccattcatgccttggtacaaattcgatgacaattatgcattcctgcaccgcacgctaaaggagattctcagaaacccgatggaggcaatgtatccacacatattctacttccactttaaaaacctacgcaaagcctatggtcggaacgaaagctggctgtgcttcaccatggaagttgtaaagcaccactcacctgtctcctggaagaggggcgtcttccgaaaccaggtggatcctgagacccattgtcatgcagaaaggtgcttcctctcttggttctgtgacgacatactgtctcctaacacaaactacgaggtcacctggtacacatcttggagcccttgcccagagtgtgcaggggaggtggccgagttcctggccaggcacagcaacgtgaatctcaccatcttcaccgcccgcctctactacttctgggatacagattaccaggaggggctccgcagcctgagtcaggaaggggcctccgtggagatcatgggctacaaagattttaaatattgttgggaaaactttgtgtacaatgatgatgagccattcaagccttggaaaggactaaaatacaactttctattcctggacagcaagctgcaggagattctcgagtgaggggtctccccgggcctcatggtctgtctcctctagcctcctgctcatgttgtgcaggcctcccctccatcctggaccagctgtgcttttgcctggtcatcctgagcccctcctggcctcagggccattccatagtgctcccctgcctcaccacctcctctccgctctcccaggctcttcctgcagaggcctctttctgcctccatggctatccatccacccaccaagaccctgttccctgagcctgcatgcccctaacctgccttttcccatctccccagcataacctaatattttttttttttttttgagacggaatttcgctctgtcacccagactggagtgcaatggcttgatcttggctcactgcaaactctgcctaccaggttcaagcgattctcctgcctccgcctcccgagtagctggaattacagacgcctgccaccacgcacagctaactttttttttttttgtatttttagtagtgactgggtttcaccatgttggccaggctggtcttgaactcctgacctcaggtgatccgcctatctcagcctcccaaagtgctgggattacaggcgtgagccactggcccggcggcacaaccaaatcttattaaactcaccctaggctggccgcggtgactcatgcctataatcccccagcaatttgggaggcagaggtgagagaatcgcttgagcccaggaattcgagaccagcctgggccacatgacaaagccccatctctacaaaaaaattacaaaaaaaaaaaaaacaggtgtggtggcatgcacctgtagttgaagctacttggaaggatgaagtgggaggattgcttgagccggggaggtggaggctgcagtgaactgagatcacgtcactgaactccagtctgagcaacagatcgagaccctgcctgaaaataaatcaataaataaactcaaccgaaatgggtatgaaagttgaaatgggtatgtaagttgaaaaccagaagttttgagaaacatcctttgttaactttcatcctacaaattgggtcattcatgtcctacgcagctaaaacagagcccaggagccagggaggaaaagcagtcaggccacacaccattgctcccaaaatggacttctctgcaagcctgactcctgaaactgtgcattgtaccctgaaaccagctttatccatagcttctgcaataaatggctgtaagtcttggactccttgctataatcgcagctattcagcaatggaacctcccagttcccaacccttcctagtgcccatgggctttcccataggacaagagaacatttctccttttctttttttttttctttgaaatggagtctcgccctgtcacccaggctggagtgcaatggtgcggtctcggctcactgcaacctctgcctcccttgttcaagtgattctcctgtctcagcctcccgagtagctgggattacaggcgtccaccaccaaaccaggctaatttttgtatttttcataaaaacgggtttcatcatgtttcccaggctggtcttatttttattttattttttgagatggagtcttgctctgttgcccaggctggggtgcagtggtgcaatctgggttcactgcagcctctgccgcctgagttcaagctattttcctacctaagcctcccaagtagctgggattacatgcgcgtgccaccacgcctagctaatttttgtgtttttagtagagacggggtttcaacatcttgaccaggctggtcttgaactcctgacctcgtgatccacccgtctcggcctcccaaagtgctgggattacaggcgtgagccacctggccaggcttaggctggtcttaaactcctgacctcaagtgatccaacctccttggcctcccaaattgctgggattgctggtgtgagccacagcgcctagcccatttctccttttaataggacctgttgctgtctctgttctcccaacatggtgaacaccacccggactgcgtgtatgtcccaaattacaattctttctttgcaaatgaaatgtgaaatttagaggcccttctccacactttaaatttgacttgacattttctaggcagatataagttattagagaatgagattctctataaaaatgatcccttcatgctgtggcctccacagaagatgccctgggccaggtgcccacatgaataatgcgggccacaggcaggcatttattttctcacagatatggaggctacaagtccaaggtggaggggtcggcggggttgtttgctctgaggccgctcctcctggatggcagggatcccttctggctgtgtcctctgtggcctttcctctatgaacctgtactgtacctctggggtctctctgcttccaaatatcttttttttttttttcagacagttttgctcttgttttctaggctggagtgcaatggcacaatctcagctcactgcaacctctgccttccgagttcaagcgattctcgtgcctcagcctcctgagtagctgggactacaggcgtgtgccaccacgcctggctaattttgtagttttagtagagacggggtttctccatgttgctcaggctggtcttgaactcatgagctcaggcgatccactctcctcagcctcccaaagtgctgggattacagatataagccaccatacacaacttttttttttttttgagatggagtttcactctgttgcccaggctggagtgctaaatagcagaatcactgctcactgcaacctctgcctgctgggttcaagcaattctcccacctcagcctcctgagtagctgggattacagatgcccagaaccaatctctgctaatttttctattttttagtagagatggggtttcactgaggaaggagaccacctctctcattgtctcctatttcagaaggaagcaaaaagttagaaagatgcagaagtaagatcaatggccagactgtttggcgctgctacctgggcctggtagttaaagatcaactcctgacctgaccgcttgttttatctaaagattccagacattgtatgaggaagcattgtgaaactttctggtctgttctgctagcccccaccactgatgcatgtagccccccagtcacgtagcccacgcttgcacaatctatcacgaccctttcacgtggaccccttagaattgtaagcccttaaaagggccagggacttcttcagggagctccaatcttcagatgcaagtctgtcaacgctcccagctgattaaagcctcttccttcctaaaaaaaaaaaaaaaaaa

SEQ ID NO: 4 - protein, 373 aa, Homo sapiens
MKPHFRNTVERMYRDTFSYNFYNRPILSRRNTVWLCYEVKTKGPSRPRLDAKIFRGQVYSQPEHHAEMCFLSWFCGNQLPAYKCFQITWFVSWTPCPDCVAKLAEFLAEHPNVTLTISAARLYYYWERDYRRALCRLSQAGARVKIMDDEEFAYCWENFVYSEGQPFMPWYKFDDNYAFLHRTLKEILRNPMEAMYPHIFYFHFKNLRKAYGRNESWLCFTMEVVKHHSPVSWKRGVFRNQVDPETHCHAERCFLSWFCDDILSPNTNYEVTWYTSWSPCPECAGEVAEFLARHSNVNLTIFTARLYYFWDTDYQEGLRSLSQEGASVEIMGYKDFKYCWENFVYNDDEPFKPWKGLKYNFLFLDSKLQEILE

SEQ ID NO: 5 - DNA, 21 bp, Artificial Sequence
  synthetically generated oligonucleotide
aaacccaaagagagaatgtga

SEQ ID NO: 6 - DNA, 35 bp, Artificial Sequence
  synthetically generated oligonucleotide
aaatctcaattcaacccaaagtaaagtaaagtaaa

(19) United States
(12) Patent Application Publication
(10) Pub. No.: US 2011/0218158 A1
(43) Pub. Date: Sep. 8, 2011

(54) DNA CYTOSINE DEAMINASE INHIBITORS

(76) Inventors: Reuben S. Harris, Saint Paul, MN (US); Ming Li, St. Paul, MN (US)

(21) Appl. No.: 13/120,381

(22) PCT Filed: Sep. 22, 2009
(86) PCT No.: PCT/US09/57832

§ 371 Date: May 27, 2011

(30) Foreign Application Priority Data
Sep. 22, 2008 (US) 61099050

(51) Int. Cl. A61K 31/7135; C12Q 1/70; A61K 31/473; A61K 31/15; A61K 31/192; A61K 31/05; A61K 31/351; A61K 31/165; A61K 31/24; A61K 31/197; A61K 31/135; A61K 31/55; A61K 31/4365; A61K 31/407; A61K 31/352; A61K 31/433; A61K 31/41; A61K 31/137; A61K 31/4164; A61P 31/18

(52) U.S. Cl. 514/23; 435/5; 514/284; 514/645; 514/570; 514/733; 514/460; 514/630; 514/538; 514/565; 514/734; 514/657; 514/217.02; 514/301; 514/410; 514/456; 514/361; 514/359; 514/653; 514/392

(57) ABSTRACT

Cytosine deaminase inhibitors and methods for identifying inhibitors of the anti-retroviral activity of APOBEC3G are described.

### CROSS-REFERENCE TO RELATED APPLICATIONS

This application claims the benefit of U.S. Provisional Application Ser. No. 61/099,050, filed Sep. 22, 2008. The disclosure of the prior application is incorporated by reference in its entirety.

### TECHNICAL FIELD

This document relates to DNA cytosine deaminase inhibitors, and more particularly to inhibitors of APOBEC3G and use of such inhibitors to reduce the anti-retroviral activity of APOBEC3G and, accordingly, reduce the HIV-1 mutation rate.

### BACKGROUND

Human APOBEC family members are important mediators of adaptive and innate immune responses. These proteins are defined by a highly conserved zinc-coordinating motif, HXE-X₂₃₋₂₈-CX₂₋₄C, in which the histidine and the two cysteines position zinc and the glutamate positions water to promote the nucleophilic deamination of cytosines within single-stranded, polynucleotide substrates (usually DNA). One family member, apolipoprotein B mRNA editing enzyme catalytic polypeptide-like 3G (APOBEC3G (A3G)), was identified as a cellular protein capable of blocking the replication of virion infectivity factor (Vif)-defective HIV-1. A3G inhibits the replication of HIV-1 and other retroviruses by deaminating viral cDNA cytosines to uracils during reverse transcription. Uracils template the incorporation of adenines during the synthesis of the complementary viral DNA strand, and subsequent replication (or DNA repair) ultimately produces strand-specific C/G to T/A transition mutations (hypermutations).

### SUMMARY

This document is based on the discovery that inhibition of APOBEC3G activity can lead to a lower HIV mutation rate and a virus population that is more stable genetically. Even a slightly lower mutation rate may render HIV more susceptible to normal adaptive immune responses. As such, compounds that inhibit the anti-retroviral activity of APOBEC3G may be effective HIV/AIDS therapeutics.

In one aspect, this document features a method for inhibiting APOBEC3G activity in a mammal (e.g., a human patient, a mouse, rat, non-human primate, or artiodactyl). The method includes administering to the mammal a therapeutically effective amount of a compound of Formula (I):

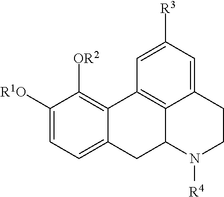

wherein:

R¹, R², and R⁴ are independently chosen from H, substituted or unsubstituted alkyl, substituted or unsubstituted alkenyl, and substituted or unsubstituted alkynyl; R³ is chosen from H and OR⁵; and R⁵ is chosen from H and substituted or unsubstituted alkyl; or a pharmaceutically acceptable salt form thereof.

For example, R¹ and R² can be H. R⁴ can be chosen from a substituted or unsubstituted alkyl and a substituted or unsubstituted alkenyl. R⁴ can be an unsubstituted alkyl such as methyl, ethyl, and propyl. R³ can be OR⁵. R⁵ can be H. The compound of Formula (I) can be chosen from:

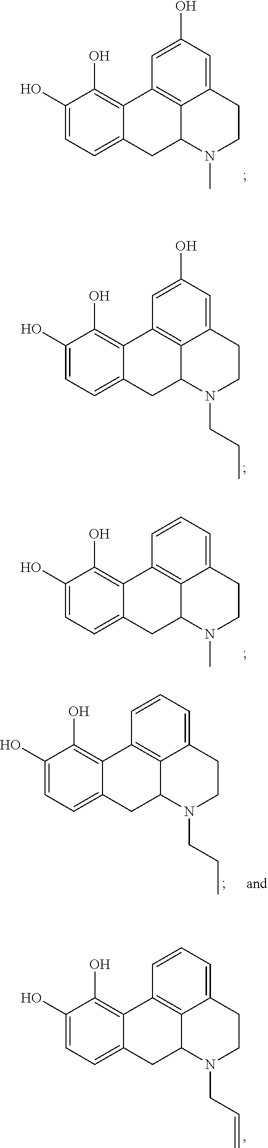

or a pharmaceutically acceptable salt form thereof.

In some embodiments, the compound of Formula (I) is chosen from:

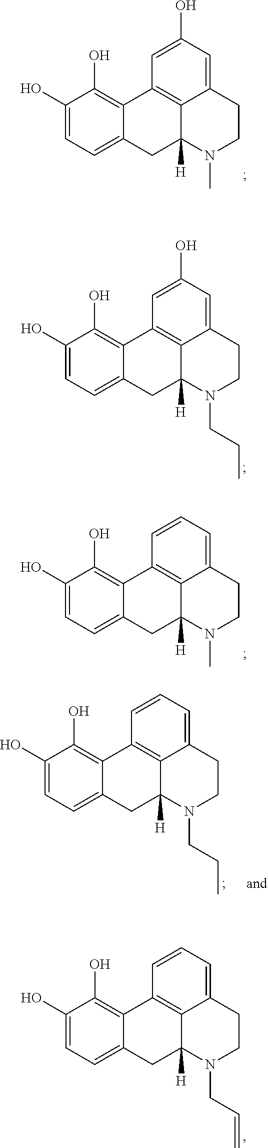

or a pharmaceutically acceptable salt form thereof.

In another aspect, this document features a method for inhibiting APOBEC3G activity in a mammal (e.g., a human patient, a mouse, rat, non-human primate, or artiodactyl). The method includes administering to the mammal a therapeutically effective amount of a compound of Formula (II):

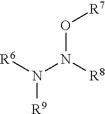

wherein:

R⁶, R⁷, and R⁸ are independently chosen from H and substituted or unsubstituted alkyl; and

R⁹ is chosen from a substituted or unsubstituted cycloalkyl and substituted or unsubstituted heterocycloalkyl;

or a pharmaceutically acceptable salt form thereof.

For example, R⁶ can be a substituted or unsubstituted alkyl such as methyl. R⁷ and R⁸ can be H. R⁹ can be a substituted or unsubstituted cycloalkyl. In some embodiments, R⁹ is chosen from 2,5-benzoquinonyl and 3,4-benzoquinonyl. A compound of Formula (II) can be chosen from:

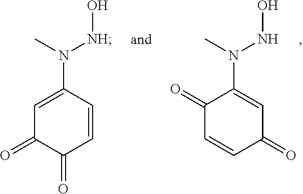

or a pharmaceutically acceptable salt form thereof.

This document also features a method for inhibiting APOBEC3G activity in a mammal (e.g., a human patient, a mouse, rat, non-human primate, or artiodactyl). The method includes administering to the mammal a therapeutically effective amount of a compound of Formula (III):

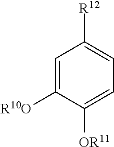

wherein:

R¹⁰ and R¹¹ are independently chosen from H and substituted or unsubstituted alkyl; and

R¹² is chosen from:

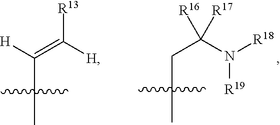

(CH₂)ₙC(O)OR²², and substituted or unsubstituted alkylaryl;

wherein:

R¹³ is chosen from C(O)NR¹⁴R¹⁵, C(O)OR¹⁴, substituted or unsubstituted cycloalkyl, substituted or unsubstituted heterocycloalkyl, substituted or unsubstituted aryl, and substituted or unsubstituted heteroaryl;

R¹⁴ is chosen from H and substituted or unsubstituted alkyl;

R¹⁵ is a substituted or unsubstituted alkylaryl;

R¹⁶ is chosen from H and substituted or unsubstituted alkyl;

R¹⁷ is chosen from H and C(O)OR²⁰;

R¹⁸ and R¹⁹ are chosen from H, C(O)R²¹, and amino;

R²⁰ and R²¹ are independently chosen from H and substituted or unsubstituted alkyl;

R²² is H or a substituted or unsubstituted alky; and

n is an integer from 1 to 6;

wherein if R¹⁶ is methyl, then at least one of R¹⁸ and R¹⁹ is not H;

or a pharmaceutically acceptable salt form thereof.

For example, R¹⁰ and R¹¹ can be H. R¹² can be

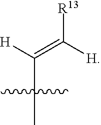

R¹³ can be C(O)OH. R¹³ can be chosen from a substituted or unsubstituted heterocycloalkyl and substituted or unsubstituted aryl. R¹² can be

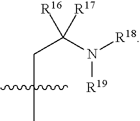

### R¹⁶ can be H. R¹⁷ can be COOH. R¹⁸ and R¹⁹ can be H.

For example, a compound of Formula (III) can be chosen from:

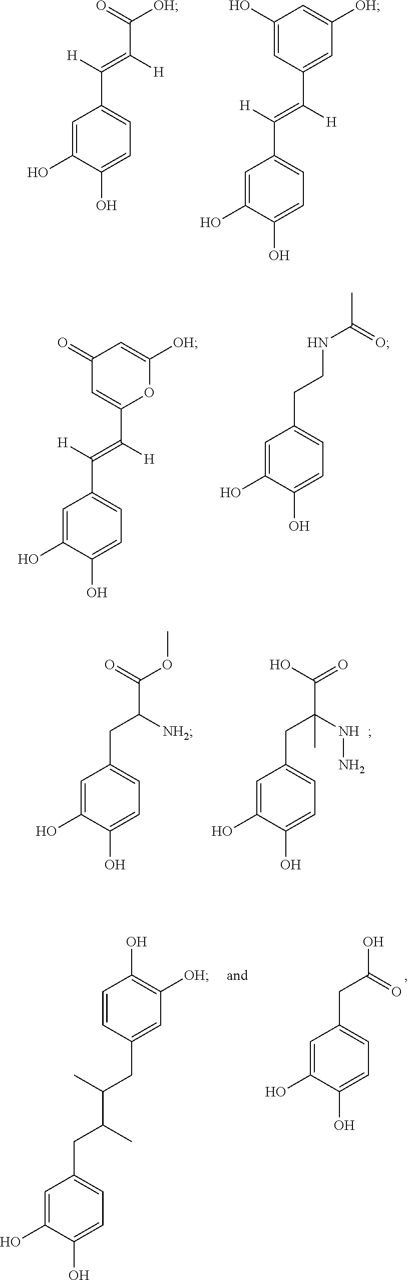

or a pharmaceutically acceptable salt form thereof.

In some embodiments, a compound of Formula (III) is:

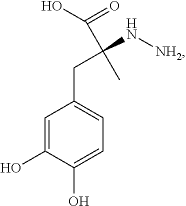

or a pharmaceutically acceptable salt form thereof.

In yet another aspect, this document features a method for inhibiting APOBEC3G activity in a mammal (e.g., a human patient, a mouse, rat, non-human primate, or artiodactyl). The method includes administering to the mammal a therapeutically effective amount of a compound of Formula (IV):

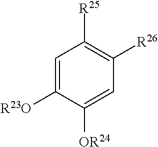

wherein:

R²³ and R²⁴ are independently chosen from H and substituted or unsubstituted alkyl;

R²⁵ is a substituted or unsubstituted heterocycloalkyl and R²⁶ is H, or R²⁵ and R²⁶ come together to form a substituted or unsubstituted cycloalkyl or a substituted or unsubstituted heterocycloalkyl;

or a pharmaceutically acceptable salt form thereof.

For example, a compound of Formula (IV) can be chosen from:

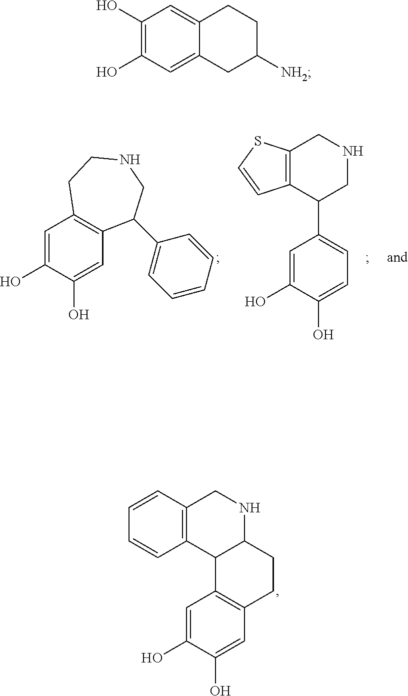

or a pharmaceutically acceptable salt form thereof.

For example, a compound of Formula (IV) can be:

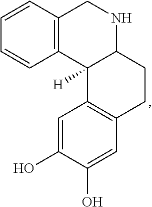

or a pharmaceutically acceptable salt form thereof.

This document also features a method for inhibiting APOBEC3G activity in a mammal (e.g., a human patient, a mouse, rat, non-human primate, or artiodactyl). The method includes administering to the mammal a therapeutically effective amount of a compound chosen from:

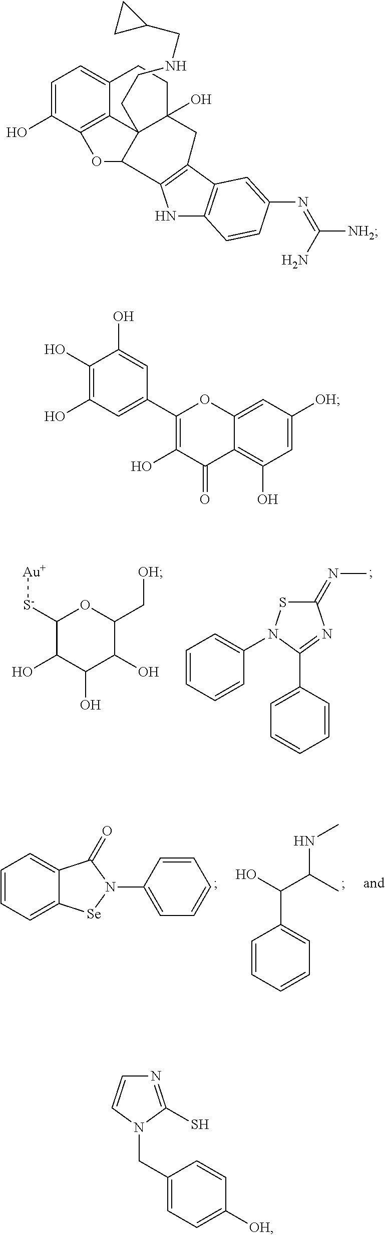

or a pharmaceutically acceptable salt form thereof.

In another aspect, this document features a method for inhibiting APOBEC3G activity in a mammal (e.g., a human patient, a mouse, rat, non-human primate, or artiodactyl). The method includes administering to the mammal a therapeutically effective amount of a compound chosen from:

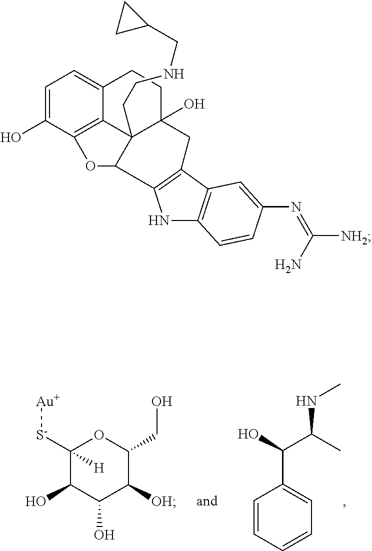

or a pharmaceutically acceptable salt form thereof.

This document also features a method of identifying inhibitors of HIV-1 mutation. The method includes measuring HIV-1 mutation rate in the presence and absence of an inhibitor of the anti-retroviral activity of APOBEC3G.

This document also features a method of identifying inhibitors of the anti-retroviral activity of APOBEC3G. The method includes assaying cytosine deaminase activity of APOBEC3G in the presence and absence of a compound, wherein the compound is identified as an inhibitor of cytosine deaminase activity if cytosine deaminase activity is reduced in the presence of the compound; and assaying replication of Vif-deficient HIV-1 in an APOBEC3G-expressing human T cell line in the presence of the inhibitor of cytosine deaminase activity, wherein the compound is identified as an inhibitor of the anti-retroviral activity of APOBEC3G if Vif-deficient HIV-1 replicates in the APOBEC3G-expressing human T cell line (e.g., CEM-SS or CEM-SS-A3G). The method further can include comparing time for Vif-proficient HIV-1 to develop drug resistance in the presence and absence of the inhibitor of cytosine deaminase activity, wherein the compound is identified as an inhibitor of the anti-retroviral activity ofAPOBEC3G when time to develop resistance is increased in the presence of the compound relative to the absence of the compound. In some embodiments, time to develop drug resistance is assessed in the presence and absence of an anti-retroviral drug. The method further can include assessing mutation profile of drug resistant Vif-proficient HIV-1. Cytosine deaminase activity can be assayed by incubating a source ofAPOBEC3G (e.g., a cell lysate or purified A3G protein), a single-strand DNA oligonucleotide substrate, and uracil DNA glycosylase in the presence and absence of the compound, wherein the oligonucleotide substrate includes a fluorescent donor molecule on its 5′ end and a fluorescent acceptor molecule on its 3′ end; and measuring fluorescence, wherein a decrease in fluorescence in the presence of the compound relative to the fluorescence in the absence of the compound indicates the compound is an inhibitor of cytosine deaminase activity.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1A is the amino acid sequence of human APOBEC3G (SEQ ID NO:2) and FIG. 1B is the nucleotide sequence (SEQ ID NO:1) encoding human APOBEC3G.

FIG. 2A is the amino acid sequence of human APOBEC3F (SEQ ID NO:4). FIG. 2B is the nucleotide sequence (SEQ ID NO:3) encoding human APOBEC3F. In FIG. 2B, the coding sequence is from nucleotides 294 to 1415.

FIG. 3 is a schematic depicting the relationship between HIV pathogenesis and mutation rate. Normally, the viral mutation rate ensures optimal levels of pathogenesis. However, both increasing (i) or decreasing (ii) the viral mutation rate is predicted to lead to a viral ‘dead zone’. An APOBEC3G-dependent increased mutation rate can cause error catastrophe (demonstrated recently by Hach& et al. (Curr Biol 18, 819-824 (2008)). A full inhibition of APOBEC3G is predicted here to increase viral genetic stability and lead to prolonged adaptive immune responses and, potentially, effective viral clearance.

FIG. 4 is a schematic of a fluorescence-based APOBEC3G DNA cytosine deamination assay. APOBEC3G can be part of a complex cytoplasmic extract (reaction requiring RNase) or provided in a pure recombinant form (no RNase requirement).

FIG. 5 contains a table of compounds that inhibited APOBEC3G activity.

FIG. 6 is a schematic of the mean relative HIV replication kinetics in virus evolution experiments. In FIG. 6A, no antiretroviral drug experimental scenarios (ART=antiretroviral therapy such as 3TC), are depicted. A-top panel: A Vif-proficient HIV is expected to grow with ‘normal’ kinetics in the absence of ART and A3G inhibitor (normal=1-2 weeks to peak replication). A-bottom panel: The addition of an A3G inhibitor is expected to ‘speed up’ the HIV replication kinetics, as the inhibitory effect of A3G on the replication of even vif-proficient viruses will be alleviated (e.g. Hache et al, 2008). In FIG. 6B, plus anti-retroviral drug experimental scenarios (ART=antiretroviral therapy such as 3TC) are depicted. B-top panel: A Vif-proficient HIV is now expected to emerge with slower kinetics in the presence of ART. The mutational pattern of such resistant viruses will consist of all types of base substitutions including an abundance of G-to-A, A3G attributable mutations. B-bottom panel: In the presence of ART, the addition of an A3G inhibitor is expected to ‘slow down’ the HIV replication kinetics. The mutational pattern of such resistant viruses can include all types of base substitutions except those of the of G-to-A type, which are expected to be significantly less frequent.

FIG. 7 is a schematic of an A3G and A3A secondary screen.

FIG. 8 is a schematic of a cell based assay (single cycle infection) of A3G inhibitors.

Like reference symbols in the various drawings indicate like elements.

### DETAILED DESCRIPTION

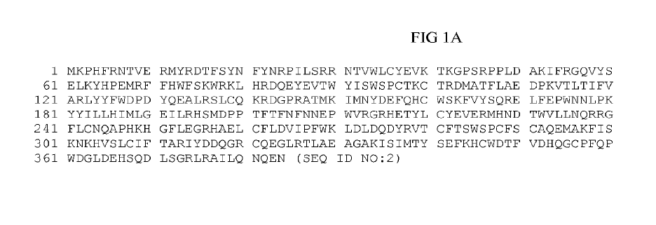

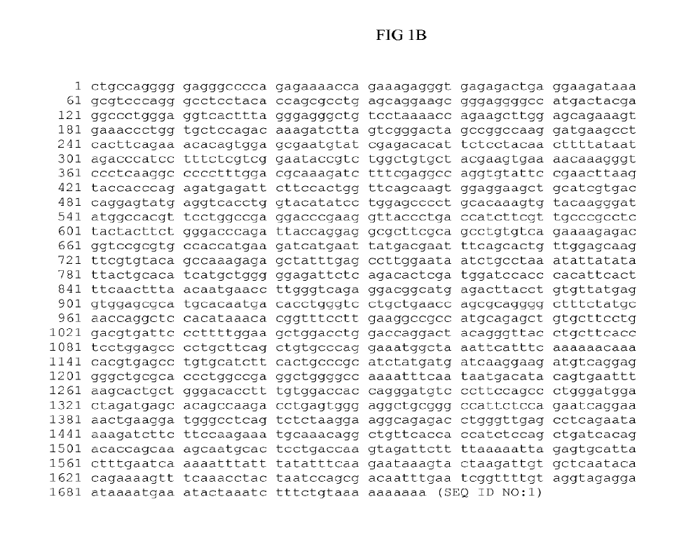

In general, this document provides inhibitors of DNA cytosine deaminases. Polypeptides having cytosine deaminase activity include single domain DNA cytosine deaminases and double domain DNA cytosine deaminases. For example, single domain DNA cytosine deaminases include, for example, activation induced deaminase (AID), APOBEC1, APOBEC2, APOBEC3A, APOBEC3C, APOBEC3D, APOBEC3E, and APOBEC3H polypeptides. Double domain DNA cytosine deaminases include, for example, APOBEC3B, APOBEC3F, and APOBEC3G polypeptides. APOBEC3D and APOBEC3E also can be produced as double domain cytosine deaminases. See, e.g., Harris and Liddament, Nat Rev Immunol. (2004), 4(11):868-77 and Jarmuz et al Genomics (2002) 79(3):285-96. APOBEC3G and/or APOBEC3F are particularly useful. Human APOBEC3G (apolipoprotein B mRNA-editing enzyme catalytic polypeptide-like 3G, also known as CEM15) uses cytosine to uracil deamination to inhibit the replication of a variety of retroviruses, including HIV-1. APOBEC3G localizes predominantly to the cytoplasm of mammalian cells. In a retrovirus-infected cell, this localization may facilitate the incorporation of APOBEC3G into viral particles, which are released from the plasma membrane. APOBEC3G also is specifically incorporated into virions through an association with the viral Gag protein and/or viral genomic RNA. Once a retrovirus enters a cell, its genomic RNA is reverse transcribed, and during this process, APOBEC3G is capable of deaminating cDNA cytosines to uracils (C->U). These lesions occur at such a high frequency that they ultimately inactivate the virus (causing G->A hypermutation, as read-out on the genomic strand of the virus). The amino acid sequence of human APOBEC3G is set forth in SEQ ID NO:2; the nucleotide sequence encoding human APOBEC3G is set forth in SEQ ID NO:1 (see FIG. 1A and 1B, respectively). See also GenBank Accession No. NM_{—}021822 for the nucleotide and amino acid sequences of human APOBEC3G.

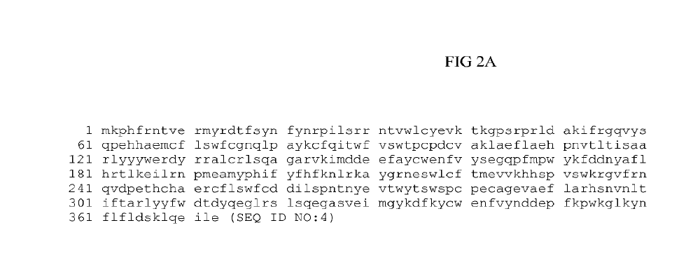

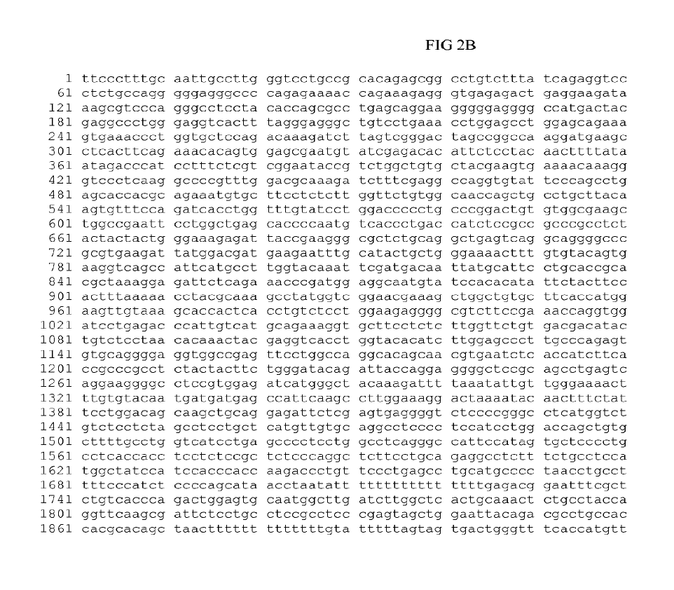

APOBEC3F is a homolog of APOBEC3G and restricts HIV-1 infection by a similar mechanism. The amino acid sequence of human APOBEC3F is set forth in SEQ ID NO:4; the nucleotide sequence encoding human APOBEC3F is set forth in SEQ ID NO:3 (see FIG. 2A and FIG. 2B, respectively). See also GenBank Accession No. NM_{—}145298 for the nucleotide sequence encoding human APOBEC3F and GenBank Accession No. NP_{—}660341 for the amino acid sequence of human APOBEC3F. APOBEC3F and -3G deaminate cytosines within different local contexts, preferring 5′-TC and 5′-CC, respectively. APOBEC3B and APOBEC3C also can restrict HIV-1 infection.

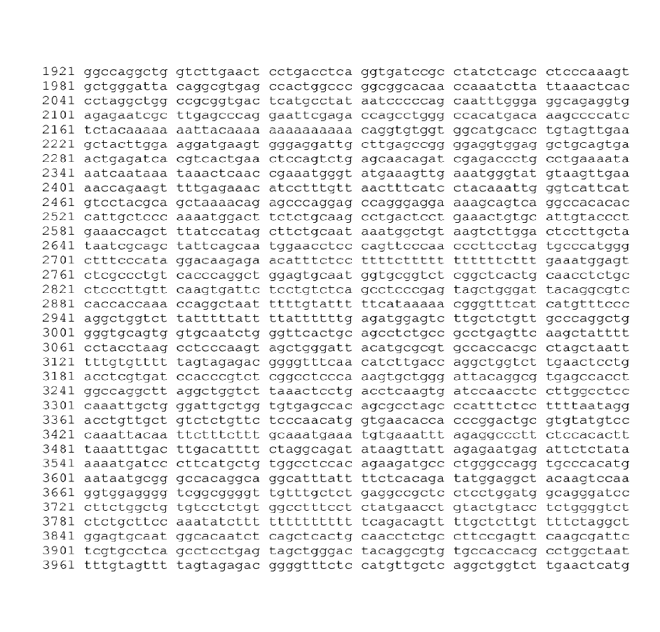

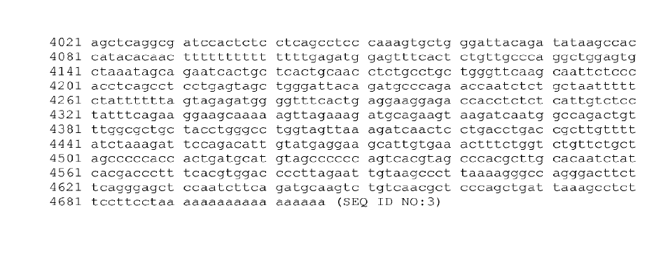

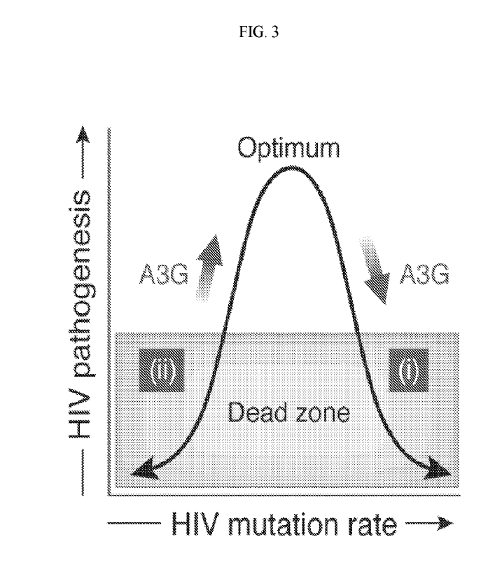

While hypermutation of retroviruses can lead to the inactivation of a virus, hypomutation also may be therapeutically useful. As such, compounds that inhibit the anti-retroviral activity of APOBEC3G (i e , inhibit cytosine deaminase activity of APOBEC3G) may be effective HIV/AIDS therapeutics. HIV can be regarded as the ultimate genetic chameleon, capable of rapidly developing resistance to anti-viral compounds, dodging adaptive immune responses and thwarting vaccine attempts. This chameleon-like nature is based on the fact that it has an extremely high mutation rate. APOBEC3G, APOBEC3F and other APOBEC3 family members contribute significantly to the optimal HIV mutation rate. For instance, there are multiple times during an infection when virion infectivity factor (Vif) incompletely neutralizes APOBEC3G: when Vif itself has a variation that promotes less effective APOBEC3G degradation, when a newly infected cell produces its first viruses, and when APOBEC3G expression is upregulated naturally. If the steady-state APOBEC3G:Vif ratio is set such that many HIV particles from infected cells contain a low, sub-lethal level of APOBEC3G that actually helps the virus achieve its optimal mutation rate (FIG. 3). Thus, it is reasonable to propose that lentiviruses use Vif purposefully to control (attenuate) the pro-mutagenic activity of the cellular APOBEC3 proteins. These ideas are supported by the fact that G-to-A mutations are the most common type of HIV genetic variation, nearly all lentiviruses have Vif and, only APOBEC3-expressing placental mammals seem to be plagued by lentiviruses. Thus, an overarching hypothesis is that HIV and other lentiviruses have become addicted to the beneficial mutations that the APOBEC3 proteins are capable of providing (beneficial from the virus' vantage). As described herein, inhibition of APOBEC3G activity can lead to a lower HIV mutation rate and a virus population that is more stable genetically. Even a slightly lower mutation rate may render HIV more susceptible to normal adaptive immune responses (antibody and cytotoxic T-cell responses) [FIG. 3 dead zone (ii)]. Thus, paradoxically, compounds that inhibit the anti-retroviral activity of APOBEC3G may be effective HIV/AIDS therapeutics.

### Definitions

For the purposes of this application, the term “aliphatic” describes any acyclic or cyclic, saturated or unsaturated, branched or unbranched carbon compound, excluding aromatic compounds.

The term “alkyl” includes straight-chain alkyl groups (e.g., methyl, ethyl, propyl, butyl, pentyl, hexyl, heptyl, octyl, nonyl, decyl, etc.) and branched-chain alkyl groups (isopropyl, tert-butyl, isobutyl, etc.). cycloalkyl (alicyclic) groups (cyclopropyl, cyclopentyl, cyclohexyl, cycloheptyl, cyclooctyl), alkyl substituted cycloalkyl groups, and cycloalkyl substituted alkyl groups. In certain embodiments, a straight chain or branched chain alkyl has 6 or fewer carbon atoms in its backbone (e.g., C₁₋₆ for straight chain, C₃₋₆ for branched chain). The term C₁₋₆ includes alkyl groups containing 1 to 6 carbon atoms.

The term “alkenyl” includes aliphatic groups containing at least one double bond and at least two carbon atoms. For example, the term “alkenyl” includes straight-chain alkenyl groups (e.g., ethylenyl, propenyl, butenyl, pentenyl, hexenyl, heptenyl, octenyl, nonenyl, decenyl, etc.) and branched-chain alkenyl groups. In certain embodiments, a straight chain or branched chain alkenyl group has 6 or fewer carbon atoms in its backbone (e.g., C₂₋₆ for straight chain, C₃₋₆ for branched chain). The term C₂₋₆ includes alkenyl groups containing 2 to 6 carbon atoms.

The term “alkynyl” includes unsaturated aliphatic groups analogous in length to the alkyls described above, but which contain at least one triple bond and two carbon atoms. For example, the term “alkynyl” includes straight-chain alkynyl groups (e.g., ethynyl, propynyl, butynyl, pentynyl, hexynyl, heptynyl, octynyl, nonynyl, decynyl, etc.) and branched-chain alkynyl groups. In certain embodiments, a straight chain or branched chain alkynyl group has 6 or fewer carbon atoms in its backbone (e.g., C₂₋₆ for straight chain, C₃₋₆ for branched chain). The term C₂₋₆ includes alkynyl groups containing 2 to 6 carbon atoms.

The term “cycloalkyl” includes a cyclic aliphatic group which may be saturated or unsatured. For example, cycloalkyl groups include cyclopropyl, cyclopentyl, cyclohexyl, cycloheptyl, and cyclooctyl. In some embodiments, cycloalkyls have from 3-12 carbon atoms in their ring structure, for example, they can have 3, 4, 5 or 6 carbons in the ring structure.

The term “heterocycloalky” includes a cyclic aliphatic group having from one to four heteroatoms.

In general, the term “aryl” includes groups, including 5- and 6-membered single-ring aromatic groups, such as benzene and phenyl. Furthermore, the term “aryl” includes multicyclic aryl groups, e.g., tricyclic, bicyclic, such as naphthalene and anthracene.

The term “heteroaryl” includes groups, including 5- and 6- membered single-ring aromatic groups, that have from one to four heteroatoms, for example, pyrrole, furan, thiophene, thiazole, isothiaozole, imidazole, triazole, tetrazole, pyrazole, oxazole, isooxazole, pyridine, pyrazine, pyridazine, and pyrimidine, and the like. Furthermore, the term “heteroaryl” includes multicyclic heteroaryl groups, e.g., tricyclic, bicyclic, such as benzoxazole, benzodioxazole, benzothiazole, benzoimidazole, benzothiophene, methylenedioxyphenyl, quinoline, isoquinoline, napthridine, indole, benzofuran, purine, benzofuran, deazapurine, indazole, or indolizine.

The term “substituted” means that an atom or group of atoms formally replaces hydrogen as a “substituent” attached to another group. For aryl and heteroaryl groups, the term “substituted”, unless otherwise indicated, refers to any level of substitution, namely mono, di, tri, tetra, or penta substitution, where such substitution is permitted. The substituents are independently selected, and substitution may be at any chemically accessible position.

As used herein, “administration” refers to delivery of a compound or composition as described herein by any external route, including, without limitation, IV, intramuscular, SC, intranasal, inhalation, transdermal, oral, buccal, rectal, sublingual, and parenteral administration.

The compounds provided herein may encompass various stereochemical forms and tautomers. The compounds also encompasses diastereomers as well as optical isomers, e.g. mixtures of enantiomers including racemic mixtures, as well as individual enantiomers and diastereomers, which arise as a consequence of structural asymmetry in certain compounds. Separation of the individual isomers or selective synthesis of the individual isomers is accomplished by application of various methods which are well known to practitioners in the art.

### Methods of IdentifYing Cytosine Deaminase Inhibitors

This document provides methods for identifying cytosine deaminase inhibitors that can be used to inhibit the anti-retroviral activity of APOBEC3G and reduce the mutation rate of HIV-1. In some embodiments, cytosine deaminase inhibitors can be used as HIV/AIDS therapeutics in mammals. In some embodiments, cytosine deaminase inhibitors can be used as chemotherapeutics. In some embodiments, cytosine deaminase inhibitors can be used in vitro and/or in vivo (e.g., in cell culture or animal models) for further understanding the biochemistry of cytosine deaminases.

In vitro assays and cell based models or in vivo models can be used to identify cytosine deaminase inhibitors. Any source of small molecules, peptides, proteins, or nucleic acids can be screened using the methods described herein. For example, any large library of chemical compounds, including libraries of natural products, libraries of synthetic compounds, or diversity oriented libraries, can be screened using the methods described herein. For example, a diversity library from ChemBridge (San Diego, Calif.) can be screened.

DNA cytosine deaminase activity can be assessed in the presence or absence of a compound using an E. coli based mutation assay. Rifampicin resistance (Rif^{R}) is attributable to base substitution mutations in the E. coli RNA polymerase B (rpoB) gene, and it occurs in approximately one of every five million bacterial cells. This assay therefore provides a robust measure of intrinsic DNA cytosine deaminase activity. See, for example, Haché et al. (2005) J Biol Chem, 280, 10920-10924; Harris et al. (2002) Molecular Cell, 10, 1247-1253.

Alternatively, a FRET based method can be used to measure cytosine deaminase activity in a high throughput manner. In such a method, a source of cytosine deaminase activity (e.g., a cell lysate or one or more purified APOBEC proteins) and a single stranded DNA oligonucleotide substrate containing an APOBEC3G deamination target site 5′CCCA (with the 3rd C strongly preferred by APOBEC3G) can be used. In some embodiments, the oligonucleotide substrate also contains an APOBEC3A deamination target site 5′TC. The 5′ end (e.g., the 5′ terminal nucleotide) of the oligonucleotide is labeled with a fluorescent donor moiety or an acceptor moiety, and the 3′ end (e.g., the 3′ terminal nucleotide) of the oligonucleotide is labeled with a fluorescent donor moiety or an acceptor moiety that is compatible with the fluorescent moiety or acceptor moiety attached to the 5′ end. A wide variety of fluorescent donors is known in the literature, including, for example, xanthene dyes, such as fluorescein or rhodamine dyes, including 5-carboxyfluorescein (FAM), 6-carboxyfluorescein (6-FAM), hexachlorofluorescein (HEX), 2′7′-dimethoxy-4′5′-dichloro-6-carboxyfluorescein (JOE), tetrachlorofluorescein (TET), fluorinated analogs of fluoresceins (e.g., Oregon Green™), 6-carboxyrhodamine (R6G), N, N, N; N′-tetramethyl-6-carboxyrhodamine (TAMRA), 6-carboxy-X-rhodamine (ROX). Suitable fluorescent donors also include the naphthylamine dyes that have an amino group in the alpha or beta position. Non-limiting examples of naphthylamino compounds include1-dimethylaminonaphthyl-5-sulfonate, 1-anilino-8- naphthalene sulfonate and 2-p-toluidinyl-6-naphthalene sulfonate, 5-(2′- aminoethyl)aminonaphthalene-1-sulfonic acid (EDANS). Other fluorescent donors include coumarins, such as 3-phenyl-7-isocyanatocoumarin; acridines, such as 9- isothiocyanatoacridine and acridine orange; N-(p-(2-benzoxazolyl) phenyl) maleimide; cyanines, such as indodicarbocyanine 3 (Cy3), indodicarbocyanine 5 (Cy5), indodicarbocyanine 5.5 (Cy5.5), 3-(-carboxy-pentyl)-3′-ethyl-5,5′- dimethyloxacarbocyanine (CyA);1H, 5H, 1 1H, 1 5H-Xantheno [2,3, 4-ij: 5,6,7-i′j′]diquinolizin-18-ium, 9-[2 (or4)-[[[6-[2, 5-dioxo-l-pyrrolidinyl) oxy]-6-oxohexyl]amino]sulfonyl]-4 (or 2)- sulfophenyl]-2,3,6,7,12,13,16,17-octahydro-inner salt (TR or Texas Red);BODIPY™ dyes;benzoxadiazoles; stilbenes; pyrenes; and the like.

An acceptor moiety must be compatible with the donor fluorescent moiety. As used herein, a “compatible acceptor” is a molecule whose absorbance spectrum overlaps with the emission spectrum of the donor. Acceptor moieties can be quenchers, i.e., a non-fluorescent molecule that causes the donor moiety to decrease its fluorescence emission intensity, or a fluorescent molecule that accepts the energy non-radiatively from the donor and re-emits the energy with the acceptor's characteristic emission spectrum. See, Epstein et al. (2002) Analytica Chimica Acta, 469:3-36.

Non-limiting examples of acceptor moieties include TAMRA, Dabcyl (4-(dimethylaminoazo)benzene-4-carboxylic acid), LC™-Red 640, LC™-Red 705, Cy5, Cy5.5 (maximally absorbs light between 500 and 705 nm), Lissamine rhodamine B sulfonyl chloride, tetramethyl rhodamine isothiocyanate, rhodamine x isothiocyanate, erythrosine isothiocyanate, fluorescein, diethylenetriamine pentaacetate or other chelates of Lanthanide ions (e.g., Europium, or Terbium). For example, 6-FAM can be used as a fluorescent donor molecule and TAMRA can be used an acceptor. Donor and acceptor moieties can be obtained, for example, from Molecular Probes (Junction City, Oreg.), Integrated DNA Technologies, Inc. (Coralville, Iowa), or Sigma Chemical Co. (St. Louis, Mo.).

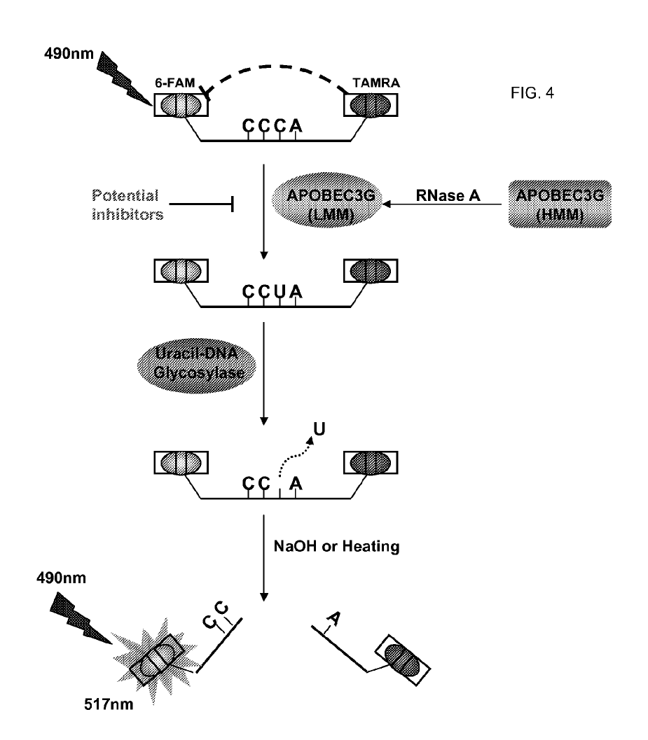

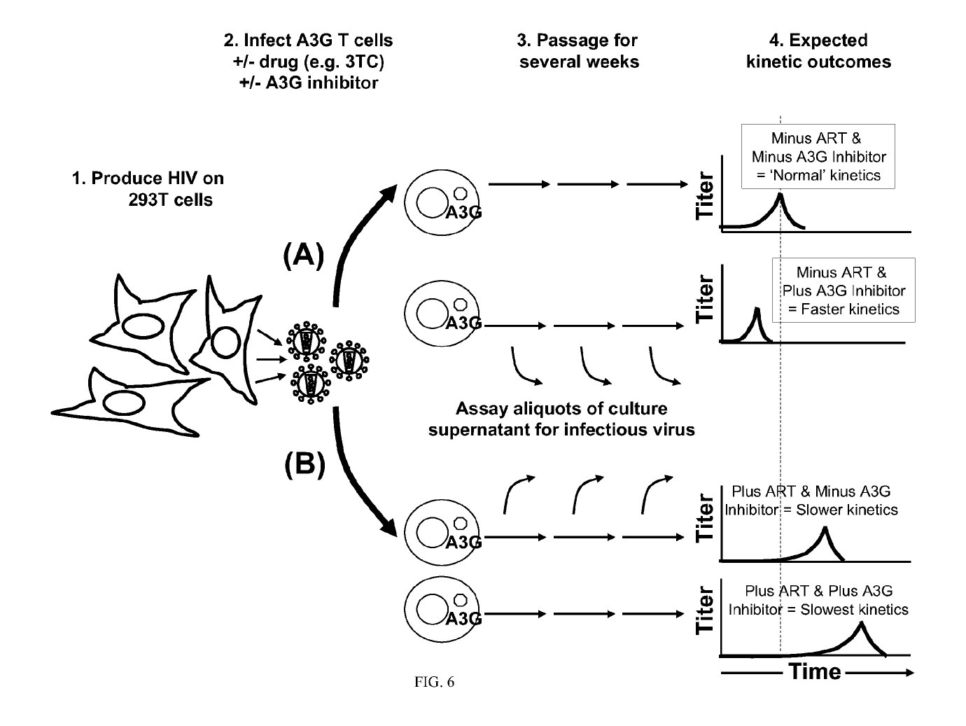

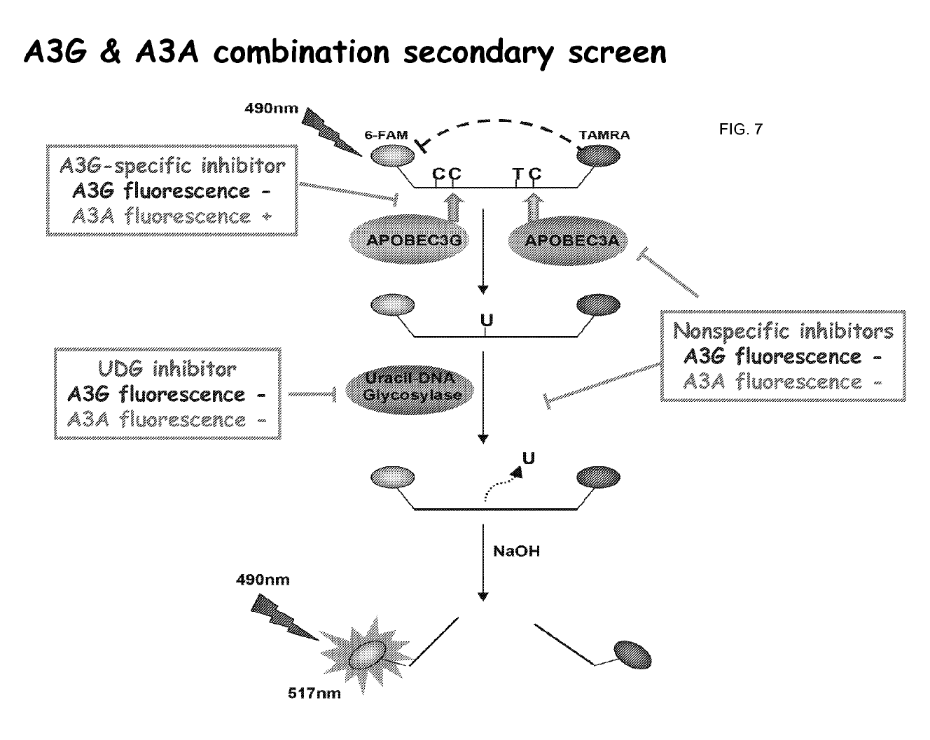

When the single strand oligonucleotide substrate has not been deaminated, the fluorescent donor and compatible acceptor moieties are in close proximity such that FRET can occur. FRET technology (see, for example, U.S. Pat. Nos. 4,996,143, 5,565,322, 5,849,489, and 6,162,603) is based on a concept that when a donor and a compatible acceptor moiety are positioned within the Förster distance of each other, energy transfer takes place between the two moieties that can be visualized or otherwise detected and/or quantitated. Förster distance refers to the distance at which resonance energy transfer between compatible FRET pairs drops to 50% and is typically between 10 and 100 Å. Thus, the intact oligonucleotide substrate emits low levels of background fluorescence because the donor molecule (e.g., 6-FAM) is quenched by the nearby acceptor (e.g., TAMRA). After the cytosine deaminase (e.g., APOBEC3G) deaminates cytosine to uracil, uracil DNA glycosylase can excise the uracil base, which creates an a-basic site that is sensitive to hydrolytic attack by the hydroxide ion of NaOH or heated water (H₂O). After treatment with NaOH or heated water, the oligonucleotide substrate is cleaved, freeing the donor molecule (e.g., 6-FAM) from inhibition by the quenching molecule (e.g., TAMRA) and yielding fluorescence (490 nm light is used to excite 6-FAM and fluorescence is detected at 517 nm). In the presence of an inhibitor of the catalytic activity of a cytosine deaminase, fluorescence is reduced. See, e.g., FIG. 4. See also FIG. 7 for a schematic of a dual APOBEC3G and APOBEC3A assay using an oligonucleotide substrate containing both APOBEC3G and APOBEC3A deamination target sites.

Methods for synthesizing oligonucleotides are known. Typically, an automated DNA synthesizer, such as available from Applied Biosystems (Foster City, Calif.), is used. Once an oligonucleotide is synthesized and any protecting groups are removed, the oligonucleotide can be purified (e.g., by extraction and gel purification or ion-exchange high performance liquid chromatography (HPLC)) and the concentration of the oligonucleotide can be determined (e.g., by measuring optical density at 260 nm in a spectrophotomer).

Oligonucleotide substrates can be labeled with donor or acceptor moieties during synthesis of the oligonucleotide or the donor or acceptor moieties can be attached after synthesis. A linker molecule can be used to attach a donor or acceptor moiety to an oligonucleotide using techniques known in the art. See, for example, The Handbook—A Guide to Fluorescent Probes and Labeling Technologies from Molecular Probes (at probes.invitrogen.com/handbook). Linkers frequently used to couple a donor fluorescent moiety such as fluorescein to an oligonucleotide include thiourea linkers (FITC-derived, for example, fluorescein-CPG's from Glen Research or ChemGene (Ashland, Mass.)), amide-linkers (fluorescein-NHS-ester-derived, such as fluorescein-CPG from BioGenex (San Ramon, Calif.)), or 3′-amino-CPG's that require coupling of a fluorescein-NHS-ester after oligonucleotide synthesis.

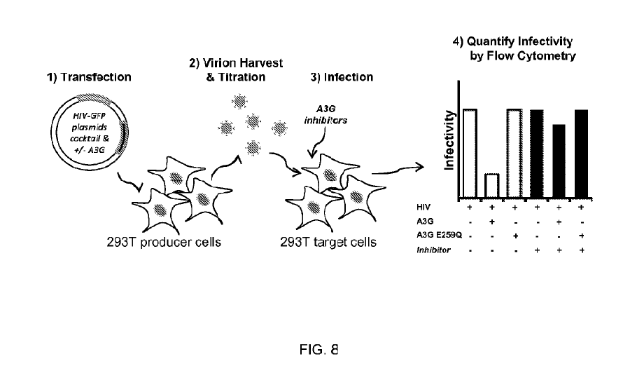

After identifying an inhibitor of cytosine deaminase activity, the toxicity profile and therapeutic efficacy of the compound can be determined by standard pharmaceutical procedures in cell culture models or animal models, including dose response curves and LD₅₀ determination. An example of a cell based assay that can be used to assess cytosine deaminase inhibitors is depicted in FIG. 8. In such a cell based assay, 293 cells can be transfected with HIV-GFP plasmids in the presence and absence of plasmids encoding A3G. After transfection, viral-like particles can be harvested and used to infect 293T target cells, which can be harvested and used to quantify infectivity by flow cytometry (Liddament et al., Curr. Biol. (2004) 14(15):1385-91. Target cells can be treated with compound before infection.

Virus growth studies also can be performed. For example, the replication of Vif-deficient HIV-1 can be assessed in an APOBEC3G expressing human T cell line (e.g., CEM-SS or CEM-SS-A3G) in the presence and absence of the compound. See, for example, the methods of Hache et al., Curr. Biol. (2008) 18:819-824. Compounds that allow replication to occur are identified as inhibitors of the anti-retroviral activity of APOBEC3G.

Virus (e.g., HIV-1) evolution studies also can be performed in the presence and absence of compound. For example, drug resistance kinetics and mutation profile of vif-proficient, pathogenic HIV-1 can be monitored in APOBEC3G expressing human T cell lines in the presence and absence of a compound and, in some embodiments, in the presence and absence of an antiretroviral (ART) drug (e.g., 3TC) (see for example, FIGS. 6A and 6B). In such experiments, the cells can be treated with a compound over a period of time (e.g., day or weeks (e.g., 4, 5, 6, 7 weeks, or longer) to allow drug resistant HIV-1 strains to be obtained. Inhibitors can decrease the viral mutation rate and therefore increase the time it takes for drug resistant strains to be detected. An additional control can include virus growth on T cells that do not express any human A3 proteins (i.e., vector control lines), where ‘faster’ growth kinetics are expected. Furthermore, the resistance mutation profile can be altered with less G-to-A mutations. Mutation profile can be assessed in any HIV-1 gene, including the gag, pol, vif, or vpr genes, by any technique. For example, DNA can be isolated from infected cells using for example, the DNeasy procedure, and a region of HIV-1 can be amplified using PCR (e.g., high-fidelity PCR). The resulting PCR products can be purified and sequenced. See HIV-1 DNA sequence analysis as set forth in Hache et al., 2008, supra.

Once a compound is determined to be effective in vitro, the compound can be tested in vivo. For example, a compound can be administered to a primate or an animal model of HIV, such as a humanized mouse model that carries human immune cells infected with the virus (see Kumar et al., Cell (2008) 134:577-586; Wege et al., Curr Top Microbiol Immunol. 2008;324:149-65.). Animals can be monitored for viral titers and CD4 counts, which positively and negatively correlate with pathogenesis. The numbers of CD4 positive T cells correlates directly with pathogenesis, with high levels correlating with protective immunity and low/no levels correlating with the immunodeficiency characteristic of AIDS. Virus DNA and RNA sequences also can be monitored periodically to assess the overall level of genetic variation. Inhibitors of APOBEC3G can cause lower overall virus titers, higher CD4 counts and little to no pathogenesis. The level of genetic variation, and in particular the level of viral G-to-A mutation can be lower. The histology of a variety of immune tissues such as spleen, lymph nodes, liver, thymus, etc also can be monitored.

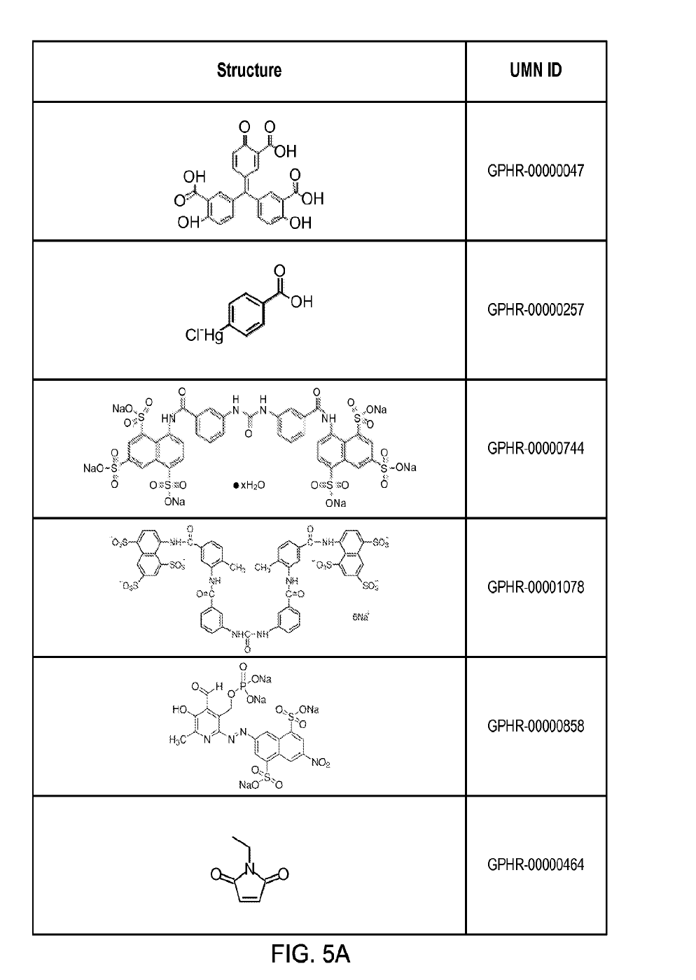

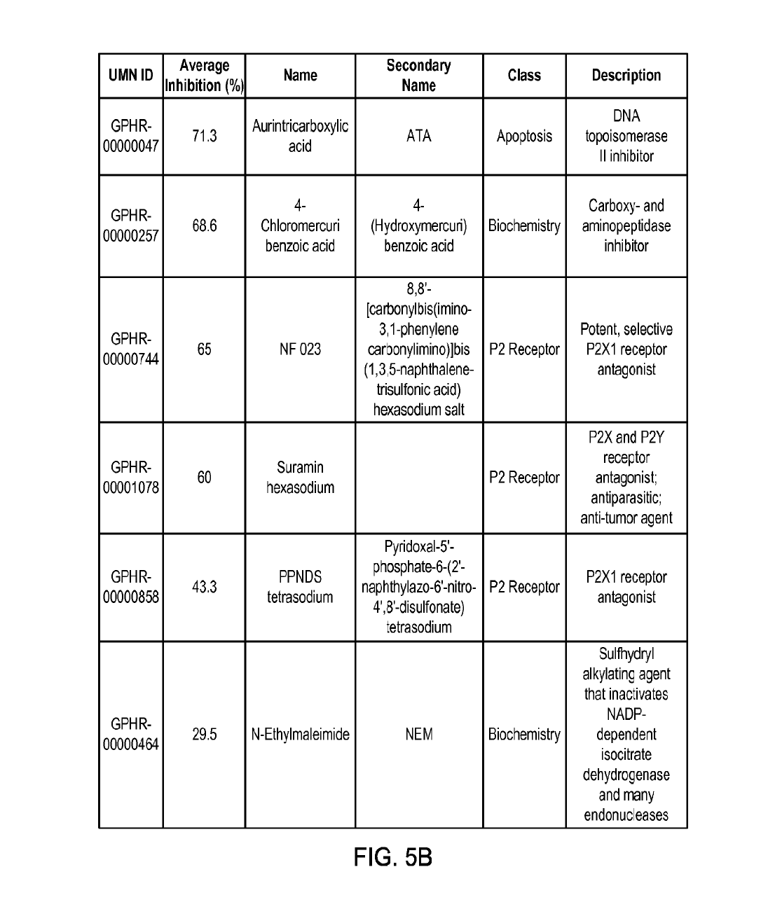

This document also provides methods for designing, modeling, and identifying compounds that can inhibit cytosine deaminase activity. Such compounds also can be referred to as “ligands” or “inhibitors.” Inhibitors of cytosine deaminase may be pyrimidine or pyrimidine-like molecules such as ATA, 4-chloromercuribenzoic acid and PPNDS (see FIG. 5). See also the compounds of Formulas (I), (II), (III), and (IV), and other compounds described herein. Such compounds may bind the active site or another portion of APOBEC3G, or interact with APOBEC3G such that its activity is inhibited.

By “modeling” is meant quantitative and/or qualitative analysis of APOBEC3G structure/function based on three-dimensional structural information. This includes conventional numeric-based molecular dynamic and energy minimization models, interactive computer graphic models, modified molecular mechanics models, distance geometry and other structure-based constraint models. Modeling typically is performed using a computer and may be further optimized using known methods.

Methods of designing ligands that bind specifically (i.e., with high affinity) to APOBEC3G (e.g., active site) typically are computer-based, and include the use of a computer having a program capable of generating an atomic model. Computer programs that use X-ray crystallography data are particularly useful for designing ligands that can interact with the active site of APOBEC. Programs such as RasMol, for example, can be used to generate a three-dimensional model of APOBEC and/or determine the structures involved in ligand binding. Computer programs such as INSIGHT (Accelrys, Burlington, Mass.), GRASP (Anthony Nicholls, Columbia University), Dock (Molecular Design Institute, University of California at San Francisco), and Auto-Dock (Accelrys) allow for further manipulation and the ability to introduce new structures.

Methods described herein can include, for example, providing to a computer the atomic structural coordinates for amino acid residues within APOBEC3G or a portion of APOBEC3G (e.g., the active site or pocket), using the computer to generate an atomic model of APOBEC3G or a portion of APOBEC3G, further providing the atomic structural coordinates of a candidate compound and generating an atomic model of a compound optimally positioned to interact with the active site of APOBEC3G, and identifying the candidate compound as a ligand of interest if the compound interacts with the active site of APOBEC3G.

Alternatively, a method for designing a ligand having specific binding affinity for the active site of APOBEC3G can utilize a computer with an atomic model stored in its memory. The atomic coordinates of a candidate compound then can be provided to the computer, and an atomic model of the candidate compound optimally positioned can be generated. For example, the three-dimensional model of APOBEC3G described by Chen et al. ((2008), Nature 452:116-119) can be used to identify inhibitors. See also U.S. Provisional Patent Application Nos. 61/063,926 and 61/085,225.

Compounds also can be interactively designed from structural information of the compounds described herein using other structure-based design/modeling techniques (see, e.g., Jackson (1997) Seminars in Oncology 24:L164-172; and Jones et al. (1996) J. Med. Chem. 39:904-917).

Compounds can be identified by, for example, identifying candidate compounds by computer modeling as interacting spatially and preferentially (i.e., with high affinity) with the active site of APOBEC3G, and then screening those compounds in vitro or in vivo for the ability to inhibit cytosine deaminase activity, allow Vif-deficient HIV-1 to replicate in APOBEC expressing human T cell lines, or increase the length of time for pathogenic HIV-1 (i.e., Vif-proficient) HIV-1 to develop resistance to a selective agent. Suitable methods for such in vitro and in vivo screening include those described herein.

Non-limiting examples of cytosine deaminase (e.g., APOBEC3G) inhibitors identified herein include compounds of Formula (I), (II), (III), and (IV). Compounds of Formula (I) have the following structure:

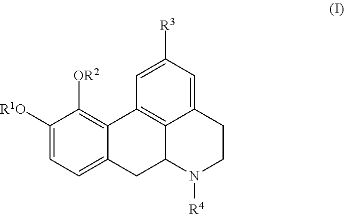

wherein:

R¹, R², and R⁴ are independently chosen from H, substituted or unsubstituted alkyl, substituted or unsubstituted alkenyl, and substituted or unsubstituted alkynyl;

R³ is chosen from H and OR⁵; and

R⁵ is chosen from H and substituted or unsubstituted alkyl;

or pharmaceutically acceptable salt forms thereof.

In some embodiments, R¹ and R² are H. In some embodiments, R⁴ is chosen from a substituted or unsubstituted alkyl and a substituted or unsubstituted alkenyl. For example, R⁴ can be an unsubstituted alkyl, such as methyl, ethyl, or propyl. In some embodiments, R³ is OR⁵. In some embodiments, R⁵ is H.

Non-limiting examples of compounds according for Formula (I) include:

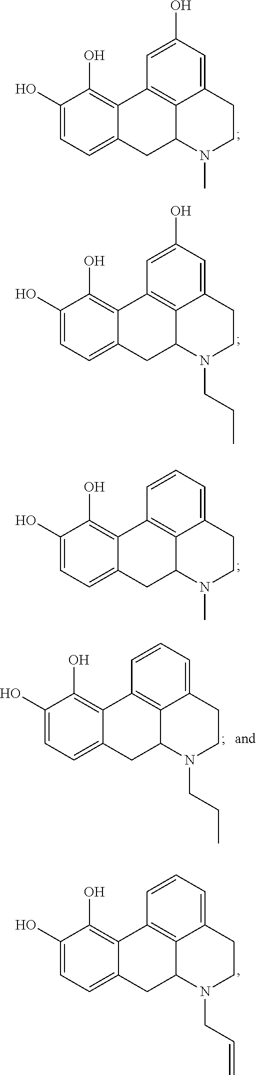

or pharmaceutically acceptable salt forms thereof. Further examples include

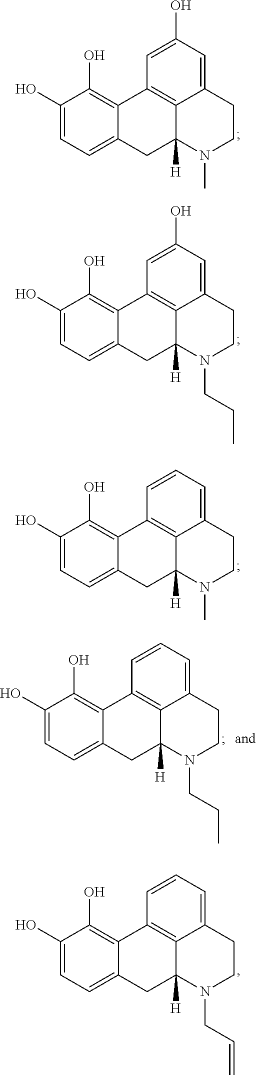

or pharmaceutically acceptable salt forms thereof.

Compounds according to Formula (II) have the following structure:

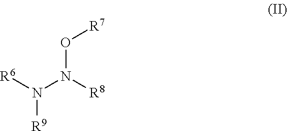

wherein:

R⁶, R⁷, and R⁸ are independently chosen from H and substituted or unsubstituted alkyl; and

R⁹ is chosen from a substituted or unsubstituted cycloalkyl and substituted or unsubstituted heterocycloalkyl;

or pharmaceutically acceptable salt forms thereof.

In some embodiments, R⁶ is a substituted or unsubstituted alkyl. For example, R⁶ can be methyl. In some embodiments, R⁷ and R⁸ are H. In some embodiments, R⁹ is a substituted or unsubstituted cycloalkyl. For example, R⁹ can be chosen from 2,5-benzoquinonyl and 3,4-benzoquinonyl.

Non-limiting examples of compounds according to Formula (II) include:

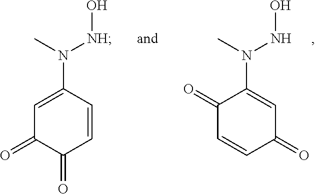

or pharmaceutically acceptable salt forms thereof.

Compounds of Formula (III) have the following structure:

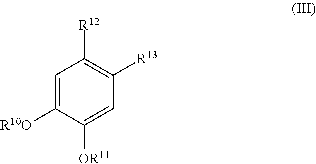

wherein:

R¹⁰ and R¹¹ are independently chosen from H and substituted or unsubstituted alkyl; and

R¹² is chosen from:

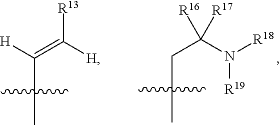

(CH₂)ₙC(O)OR²², and substituted or unsubstituted alkylaryl;

wherein:

R¹³ is chosen from C(O)NR¹⁴R¹⁵, C(O)OR¹⁴, substituted or unsubstituted cycloalkyl, substituted or unsubstituted heterocycloalkyl, substituted or unsubstituted aryl, and substituted or unsubstituted heteroaryl;

R¹⁴ is chosen from H and substituted or unsubstituted alkyl;

R¹⁵ is a substituted or unsubstituted alkylaryl;

R¹⁶ is chosen from H and substituted or unsubstituted alkyl;

R¹⁷ is chosen from H and C(O)OR²⁰;

R¹⁸ and R¹⁹ are chosen from H, C(O)R²¹, and amino;

R²² and R²¹ are independently chosen from H and substituted or unsubstituted alkyl;

R²² is H or a substituted or unsubstituted alky; and

n is an integer from 1 to 6;

wherein if R¹⁶ is methyl, then at least one of R¹⁸ and R¹⁹ is not H;

or pharmaceutically acceptable salt forms thereof.

In some embodiments, R¹⁰ and R¹¹ are H. In some embodiments, R¹² is

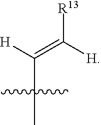

In some embodiments, R¹³ is C(0)OH or R¹³ is chosen from a substituted or unsubstituted heterocycloalkyl and substituted or unsubstituted aryl. In other embodiments, R¹² is

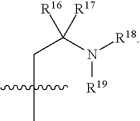

In some embodiments, R¹⁶ is H. In some embodiments, R¹⁷ is COOH. In some embodiments, R¹⁸ and R¹⁹ are H.

Non-limiting examples of compounds according to Formula (III) include:

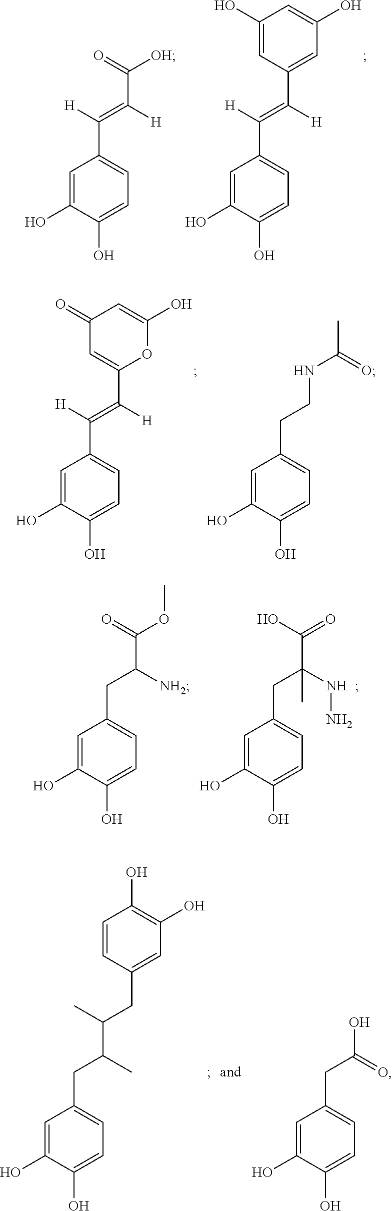

or pharmaceutically acceptable salt forms thereof. For example, the compound can be

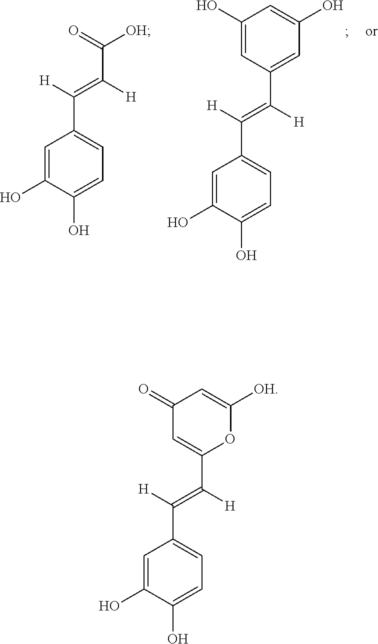

A further example includes:

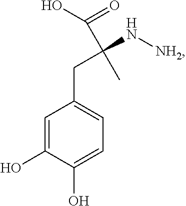

or a pharmaceutically acceptable salt form thereof.

Also described herein are compounds according to Formula (IV):

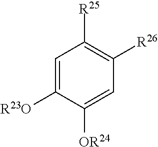

wherein:

R²³ and R²⁴ are independently chosen from H and substituted or unsubstituted alkyl;

R²⁵ is a substituted or unsubstituted heterocycloalkyl and R²⁶ is H, or R²⁵ and R²⁶ come together to form a substituted or unsubstituted cycloalkyl or a substituted or unsubstituted heterocycloalkyl;

or pharmaceutically acceptable salt forms thereof.

Non-limiting examples of compounds according to Formula (IV) include:

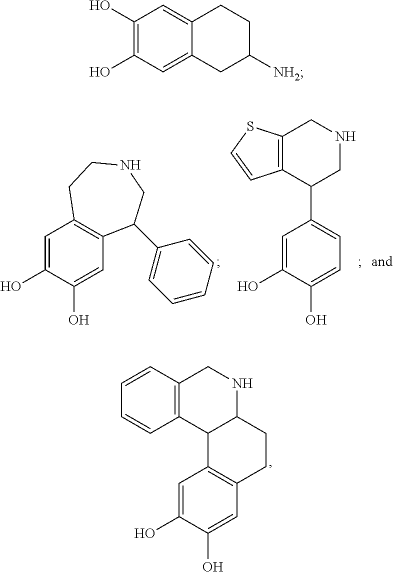

or pharmaceutically acceptable salt forms thereof. A further example includes:

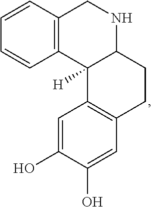

or a pharmaceutically acceptable salt form thereof.

Other examples of cytosine deaminase inhibitors include compounds:

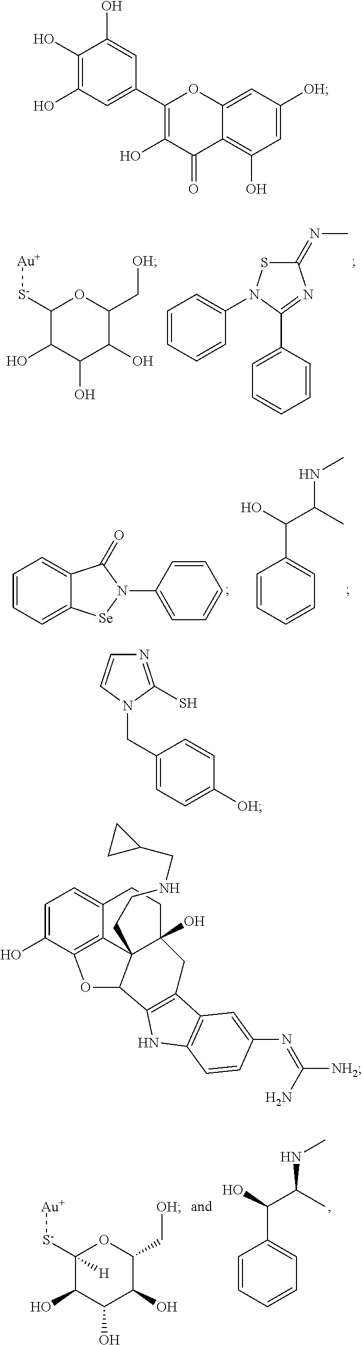

or pharmaceutically acceptable salt forms thereof

Pharmaceutically acceptable salts of the compounds described herein include the acid addition and base salts thereof. Suitable acid addition salts are formed from acids which form non-toxic salts. Examples include the acetate, adipate, aspartate, benzoate, besylate, bicarbonate/carbonate, bisulphate/sulphate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, hydrogen phosphate, isethionate, D- and L-lactate, malate, maleate, malonate, mesylate, methylsulphate, 2-napsylate, nicotinate, nitrate, orotate, oxalate, palmitate, pamoate, phosphate/hydrogen, phosphate/phosphate dihydrogen, pyroglutamate, saccharate, stearate, succinate, tannate, D- and L-tartrate, 1-hydroxy-2-naphthoate tosylate and xinafoate salts.

Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts.

Hemisalts of acids and bases may also be formed, for example, hemisulphate and hemicalcium salts.

A person skilled in the art will know how to prepare and select suitable salt forms for example, as described in “Handbook of Pharmaceutical Salts: Properties, Selection, and Use” by Stahl and Wermuth (Wiley-VCH, Weinheim, Germany, 2002).

The compounds for use in the compositions and methods provided herein may be obtained from commercial sources (e.g., Aldrich Chemical Co., Milwaukee, Wis.) or may be prepared by methods well known to those of skill in the art.

### Pharmaceutical Compositions

Compounds that inhibit cytosine deaminase activity, including compounds of Formula (I), (II), (III), and (IV), can be used in vitro (e.g., in cell culture) or administered to a mammal (e.g., a human patient, non-human primates such as monkeys, baboons, or chimpanzees, horses, artiodactyls such as cows (cattle or oxen), pigs, sheep, or goats, cats, rabbits, guinea pigs, hamsters, rats, gerbils, and mice) to inhibit cytosine deaminase activity of the mammal. Compounds described herein can be administered by any route, including, without limitation, oral or parenteral routes of administration such as intravenous, intramuscular, intraperitoneal, subcutaneous, intrathecal, intraarterial, nasal, transdermal (e.g., as a patch), or pulmonary absorption. Cytosine deaminase inhibitors can be administered alone or in combination with one or more other compounds described herein or in combination with one or more other drugs (or as any combination thereof). For example, two or more compounds identified by the methods described herein can be administered to a mammal. In some embodiments, one or more cytosine deaminase inhibitors are administered to a mammal (e.g., a human patient, mouse, rat, non-human primate, or artiodactyl) in combination with an HIV-1/AIDS therapeutic. Non-limiting examples of HIV-1/AIDS therapeutics include reverse transcriptase inhibitors such as a nucleoside analog reverse transcriptase inhibitor (e.g., Zidovudine, Didanosine, Zalcitabine, Stavudine, Lamivudine, Abacavir, Emtricitabine), nucleotide analog reverse transcriptase inhibitor (e.g., Tenofovir or Adefovir), or a non-nucleoside reverse transcriptase inhibitor (e.g., Efavirenz, Nevirapine, Delavirdine, or Etravirine); protease inhibitors (e.g., Saquinavir, Ritonavir, Indinavir, or Nelfinavir); entry/fusion inhibitors; integrase inhibitors (e.g., Raltegravir or Elvitegravir); maturation inhibitors; or portmanteau inhibitors (drug that is a combination of two drug molecules, each of which is itself a type of inhibitor, e.g., reverse transcriptase inhibitor and an integrase inhibitor).

The compounds described herein may be administered as crystalline or amorphous products. They may be obtained, for example, as solid plugs, powders, or films by methods such as precipitation, crystallization, freeze drying, spray drying, or evaporative drying. Microwave or radio frequency drying may be used for this purpose.

Generally, they will be administered as a formulation in association with one or more pharmaceutically acceptable excipients. The term “excipient” is used herein to describe any ingredient other than the compound(s) of the invention. The choice of excipient will to a large extent depend on factors such as the particular mode of administration, the effect of the excipient on solubility and stability, and the nature of the dosage form. Non-limiting examples of pharmaceutical excipients suitable for administration of the compounds provided herein include any such carriers known to those skilled in the art to be suitable for the particular mode of administration. Pharmaceutically acceptable excipients include, but are not limited to, ion exchangers, alumina, aluminum stearate, lecithin, self-emulsifying drug delivery systems (SEDDS) such as d-α-tocopherol polyethylene glycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium-chloride, zinc salts, colloidal silica, magnesium trisilicate, polyvinyl pyrrolidone, cellulose-based substances, polyethylene glycol, sodium carboxymethyl cellulose, polyacrylates, waxes, polyethylene-polyoxypropylene-block polymers, and wool fat. Cyclodextrins such as α-, β, and γ-cyclodextrin, or chemically modified derivatives such as hydroxyalkylcyclodextrins, including 2- and 3-hydroxypropyl-b-cyclodextrins, or other solubilized derivatives can also be advantageously used to enhance delivery of compounds of the formulae described herein. Preservatives, flavorings, sugars, and other additives such as antimicrobials, antioxidants, chelating agents, inert gases, and the like also may be present. In some embodiments, the excipient is a physiologically acceptable saline solution.

The compositions can be, in one embodiment, formulated into suitable pharmaceutical preparations such as solutions, suspensions, emulsions, tablets, dispersible tablets, pills, capsules, powders, sustained release formulations or elixirs, for oral administration or in sterile solutions or suspensions for parenteral administration, as well as transdermal patch preparation and dry powder inhalers (see, e.g., Ansel Introduction to Pharmaceutical Dosage Forms, Fourth Edition 1985, 126). For oral administration, tablets or capsules can be prepared by conventional means with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g. magnesium stearate, talc or silica); disintegrants (e.g., potato starch or sodium starch glycolate); or wetting agents (e.g., sodium lauryl sulfate). Tablets can be coated by methods known in the art. Preparations for oral administration can also be formulated to give controlled release of the compound.

Nasal preparations can be presented in a liquid form or as a dry product. Nebulised aqueous suspensions or solutions can include carriers or excipients to adjust pH and/or tonicity.

The concentration of a compound in a pharmaceutical composition will depend on absorption, inactivation and excretion rates of the compound, the physicochemical characteristics of the compound, the dosage schedule, and amount administered as well as other factors known to those of skill in the art.

The pharmaceutical composition may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular patient, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

The pharmaceutical compositions can be provided for administration to humans and animals in unit dosage forms, such as tablets, capsules, pills, powders, granules, sterile parenteral solutions or suspensions, and oral solutions or suspensions, and oil-water emulsions containing suitable quantities of the compounds or pharmaceutically acceptable derivatives thereof. The pharmaceutically therapeutically active compounds and derivatives thereof are, in one embodiment, formulated and administered in unit-dosage forms or multiple-dosage forms. Unit-dose forms as used herein refers to physically discrete units suitable for human and animal patients and packaged individually as is known in the art. Each unit-dose contains a predetermined quantity of the therapeutically active compound sufficient to produce the desired therapeutic effect, in association with the required pharmaceutical carrier, vehicle or diluent. Examples of unit-dose forms include ampoules and syringes and individually packaged tablets or capsules. Unit-dose forms may be administered in fractions or multiples thereof. A multiple-dose form is a plurality of identical unit-dosage forms packaged in a single container to be administered in segregated unit-dose form. Examples of multiple-dose forms include vials, bottles of tablets or capsules or bottles of pints or gallons. Hence, multiple dose form is a multiple of unit-doses which are not segregated in packaging.

Liquid pharmaceutically administrable compositions can, for example, be prepared by dissolving, dispersing, or otherwise mixing an active compound as defined above and optional pharmaceutical adjuvants in a carrier, such as, for example, water, saline, aqueous dextrose, glycerol, glycols, ethanol, and the like, to thereby form a solution or suspension. If desired, the pharmaceutical composition to be administered may also contain minor amounts of nontoxic auxiliary substances such as wetting agents, emulsifying agents, solubilizing agents, pH buffering agents and the like, for example, acetate, sodium citrate, cyclodextrine derivatives, sorbitan monolaurate, triethanolamine sodium acetate, triethanolamine oleate, and other such agents.

Dosage forms or compositions containing a compound as described herein in the range of 0.005% to 100% with the balance made up from non-toxic carrier may be prepared. Methods for preparation of these compositions are known to those skilled in the art. The contemplated compositions may contain 0.001%-100% active ingredient, in one embodiment 0.1-95%, in another embodiment 75-85%.

Pharmaceutical compositions suitable for the delivery of compounds described herein and methods for their preparation will be readily apparent to those skilled in the art. Such compositions and methods for their preparation may be found, for example, in “Remington's Pharmaceutical Sciences”, 19th Edition (Mack Publishing Company, 1995).

### Articles of Manufacture

Compounds inhibiting cytosine deaminase activity (e.g., compounds of Formulas (I), (II), (III), (IV), or other compounds described herein) and pharmaceutical compositions containing such compounds can be combined with packaging material and sold as articles of manufacture for inhibiting cytosine deaminase activity, reducing HIV-1 mutation rate, or treating HIV-1/AIDS. The articles of manufacture may combine one or more compounds identified using the in vitro or in vivo methods as described herein. In addition, the articles of manufacture may further include reagents such as buffers, additional HIV-1/AIDS therapeutics, or other useful reagents for assessing cytosine deaminase activity, inhibiting cytosine deaminase activity, reducing HIV-1 mutation rate, or treating HIV-1/AIDS. Instructions describing how the various reagents are effective for inhibiting cytosine deaminase activity, reducing HIV-1 mutation rate, or treating HIV-1/AIDS also may be included in such kits.

The invention will be further described in the following examples which do not limit the scope of the invention described in the claims.

### EXAMPLES

### Example 1

### Fluorescence-Based APOBEC3G-DNA Cytosine Deaminase Assay for High Throughput Screening (HTS)

FIG. 4 contains a schematic of a fluorescence based APOBEC3G DNA cytosine deaminase activity assay. The single-strand DNA oligonucleotide substrate has 3 features: i) an APOBEC3G deamination target cite 5′CCCA (with the 3rd C strongly preferred by APOBEC3G), ii) a fluorescent 6-FAM label on the 5′ end (excitable by 490 nm wavelength light) and iii) a 6-FAM ‘quenching’ molecule TAMRA on the 3′ end. This intact ssDNA substrate emits low levels of background fluorescence because the 6-FAM is quenched by the nearby TAMRA. Low-molecular mass (LMM) APOBEC3G, derived from an RNase sensitive high-molecular mass (HMM) cellular complex, efficiently deaminates the indicated cytosine base yielding a uracil. Uracil DNA glycosylase excises the uracil base, which creates an a-basic site that is sensitive to hydrolytic attack by the hydroxide ion of NaOH or heated water (H2O). Finally, cleavage of the ssDNA frees 6-FAM from inhibition by TAMRA and results in fluorescence (490 nm light is used to excite 6-FAM and fluorescence is detected at 517 nm).

APOBEC3G containing cell lysates were prepared from near-confluent APOBEC3G expressing 293 stable cell line (211-21, Harris et al., Cell 2003). DMEM growth medium was aspirated and the cells suspended in 1xPBS, 1 mM EDTA, pH 7.4. The cells were pelleted at 1.2K rpm for 5-10 minutes. The supernatant was aspirated and the tube containing the pelleted cells was placed on ice. Thirty mL of lysis buffer (25 mM HEPES, pH7.4, 150 mM NaCl, 0.5% Triton X-100, 1 mM EDTA, 1 mM MgCl₂, 1 mM ZnCl₂, 10% Glycerol, and Roche EDTA free complete protease inhibitor (added before use)) was added per 3-5 g cell pellet along with 20 μg/mL RNase A (from Qiagen, catalog no. 19101, 100 mg/ml RNase A solution) and the cells suspended by pipetting. The cells were transferred to a cold homogenizer and homogenized for 10 strokes. After homogenization, the cells were incubated on ice for 60 min with periodic vortexing or rotating then incubated at room temperature for another 20min. The cells were spun at 15,000 rpm, 4° C. for 10 min and the supernatant harvested. The protein concentration in the cell lysate was quantified using a Bio-Rad protein assay kit or DC protein assay kit, following the manufacturer's instructions.

A fluorescence-based deaminase activity assay for HTS was performed as follows: 1) 20 μl of the crude cell lysate (15 μg protein, diluted in cell lysis buffer, pH7.4) was placed into 384 well plates using Perkin Elmer flexdrop. 2) DMSO (40 nl), chemicals (in 40 nl DMSO) and uracil-DNA glycosylase (UDG) (0.02 NEB units of UDG) were added using ECHO 550, mixed by shaking, and incubated at 37° C. for 15 min. UDG, 5000 units/mL, was obtained from NE Biolab, catalog no. MO280S. Twenty (20) μl of an oligo mix (10 pmol oligo, 2 μg RNase A in 50 mM Tris.HCl, pH7.4, 10 mM EDTA, diluted from 100 μM oligo stock and 100 mg/ml RNase A stock) using Perkin Elmer flexdrop, mixed by shaking, and incubated at 37° C. for 2 hrs. The oligonucleotide was a 6-FAM and TAMRA dual-labeled 2lmer DNA oligo (AAACCCAAAGAGAGAATGTGA; SEQ ID NO:5) in water (Biosearch). 4) 3 μl of 4N NaOH was added using Perkin Elmer flexdrop, mixed by shaking and incubated at 37° C. for 30 min. 5) 3 μl N HCl+27 μl 2 M Tris.Cl (pH7.9) was added using Perkin Elmer flexdrop, mixed by shaking, and fluorescence read with excitation at 492 nm and emission at 517 nm at room temperature in LJL Analyst AD.

### Example 2

### Screening Library for APOBEC3G Catalytic Inhibitors

A 1280 compound library (LOPAC¹²⁸⁰™, from Sigma-Aldrich) was screened in quadruplicate using the method set forth in Example 1. As shown in FIG. 5, six compounds were identified that inhibited cytosine deaminase activity. The screen of this library was repeated with an additional step at the beginning during which the cell lysate used as a source of A3G was incubated with a 10 μM solution of the compound to be tested dissolved in DMSO. In these experiments, each compound was evaluated a total of four times—twice on one day and twice more on the following day. A total of 5 primary actives, where a primary active was taken as at least 35% inhibition of fluorescence relative to positive controls, were identified when this screen was repeated. One of the compounds identified in this screen was suramin (MW 1297.3), which is probably an RNase inhibitor since it is a well-known promiscuous enzyme inhibitor. While this is not likely to be useful for studying A3G activity in cells, its identification confirmed the effectiveness of the screening method. Furthermore, suramin could be used as a control for subsequent HTS since there is no true control for the inhibition of A3G deaminase activity.

### Example 3

### Using Purified A3G Protein in Screening Assays

Using a purified form of active low molecular weight A3G obviates the need for RNase A in the deaminase assay since the reason for including RNase A is to free A3G from the inactive high molecular mass complexes it forms in some cells. A3G protein was purified as follows. Briefly, 5 g of 293 cells stably expressing A3G-Myc-His were prepared in 30 mL of lysis buffer according to the cell lysate preparation described above. To this lysate, 200 μl of nickel-nitriloacetic acid (Ni-NTA) agarose (Qiagen) were added and mixed on an end-over-end shaker overnight at 4° C. before loading onto a standard chromatography column (Bio-Rad). After washing according to manufacturer's instructions (50 mM Tris, pH 8.0, 0.3 M NaCl, 10% glycerol with 30-50 mM imidazole), bound proteins were eluted 4×100 μL in wash buffer containing 120 mM imidazole. Protein purity and quantity were determined by Coomassie Blue staining, anti-Myc Western blot, anti-A3G Western blot and the Bradford Assay (Bio-Rad). Using this protocol, approximately 80% pure recombinant A3G-myc-his was isolated from HEK-293 cells stably expressing this protein. The activity of this protein is RNase-independent. The recombinant A3G-myc-his protein was found to be amenable to the HTS 384 well plate format described in Example 1 using only 60 ng of the recombinant protein, 0.02 units of E. coli UDG (New England Biolabs), 10 pmol substrate oligonucleotide, and 40 nl DMSO in 40 μl of total reaction volume. Repeating the primary screen of the 1280 compound LOPAC¹²⁸⁰™ library using pure A3G yielded 36 actives with >50% inhibition of A3G. Activity of the 36 compounds was re-confirmed in A3G dose response testing.

### Example 4

### Secondary Screens of A3G Inhibitors Using A3A

To ensure that the compounds identified in Example 3 were specific A3G inhibitors, a secondary screen was performed using the related zinc-dependent DNA deaminase APOBEC3A (A3A). The elegance of a secondary screen with recombinant A3A is that it eliminates most nonspecific enzyme inhibitors such as cross-linking compounds, DNA binding compounds, fluorescence quenching agents, metal chelators, etc. It also eliminates compounds that inhibit Uracil DNA Glycosylase because A3A also requires this enzyme to yield fluorescence signal in the assay.

A3A-Myc-His was prepared from transiently transfected human 293 cells using the same protocol as described in Example 3. The resulting A3A enzyme was similarly pure and highly active. The assay of Example 1 was repeated with the 36 compounds identified in the primary screen, purified A3A and A3G enzymes, and a substrate having both an A3G-specific mutational site and an A3A-specific mutational site to avoid biasing results by the inherent nucleotide mutational preferences of each APOBEC3 protein (5′-FAM -AAATCTCAATTCAACCCAAAGTAAAGTAAAGTAAA—TAMRA (Biosearch Technologies, Inc., SEQ ID NO:6). A schematic of the assay is depicted in FIG. 7. Of the 36 compounds identified in the primary screen, 25 were A3G specific (i.e., inhibited A3G, but not A3A or UDG), two had dual specificity (i e , inhibited A3A and A3G but not UDG), and 9 were non-specific (i.e., inhibited A3A, A3G, and UDG). The 25 A3G specific compounds and the two compounds with dual specificity had IC₅₀s≦10 μM with A3G and IC₅₀s of ≧50 μM with A3A when assessed using the HTS platform. Similarly, when the dose response experiments were repeated in the laboratory with manual pipetting instead of the HTS platform, IC50s of ≦8 μM were obtained with A3G and IC₅₀s of >20 μM were obtained with A3A.

### Example 5

### Compound Secondary Toxicity Screen

Each compound displaying specificity for A3G is tested for cytotoxicity. This is done by incubating each inhibitor with several model T cell lines (CEM, H9, SupT1, Jurkat, etc). Following incubation for two days with a given compound at concentrations covering a range centered on the IC₅₀, cells are assessed for viability by staining with the viability exclusion dye propidium iodide and assessed by flow cytometry. Results are used to calculate an LC₅₀.

### Example 6

### Cell Based Assay (Single Cycle Infection)

HIV-GFP virus stocks were produced by transfected 293T cells with Transit-LT1 transfection reagent and the following plasmid cocktail:pCS-CG, pRK5/Pack1 (Gag-Pol), pRK5/Rev, pMDG (vesicular stomatitis virus G protein), together with an empty vector control, or expression constructs for APOBEC3G or APOBEC3G-E259Q (catalytically deficient A3G). Forty-eight (48) hours post-transfection, viral-like particles containing supernatants were harvested, titrated and use to infect 293T cells. After an additional 48 hours, 293T cells were harvested and used to quantify infectivity by flow cytometry (Liddament et al., 2004, supra). Target cells were treated with 100 μM of compound two hours before infection. HIV-GFP infectivity is expressed as the % of GFP positive target cells.

### Example 7

### Cell-Based Validation of Candidate Compound Efficacy

Candidate compounds with acceptable therapeutic indices are evaluated in cell culture experiments. Specifically, A3G inhibitors are incubated with A3Gexpressing CEM-SS and SupT1 cells, and these cells will be infected with ΔVif HIV. Cells expressing wild-type A3G restrict the growth of ΔVif HIV, while the same virus grows on cells expressing catalytically deficient A3G E259Q. If these inhibitors decrease A3G deaminase activity, they should therefore cause treated wild-type A3G-expressing cells to no longer restrict ΔVif HIV, reproducing the phenotype of ΔVif HIV growth on control A3G E259Q-expressing cells.

### Example 8

### Virus Production, Titer and Monitoring

HIV are produced by transfecting 293T cells with proviral plasmids encoding replication-competent AVif HIV viruses and wild-type controls. Virus-containing supernatants are harvested and purified through 0.45 μm filters, and stocks are titered by infecting CEM-GFP indicator cells, which glow green upon infection with HIV. Quantification of green cells in culture three days post-infection will therefore permit the calculation of a multiplicity of infection (MOI). Normalizing by MOI, cells are infected and viral growth monitored over time by periodic infection of CEM-GFP cells with virus containing supernatants from the experimental cultures.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for inhibiting APOBEC3G activity in a mammal, said method comprising administering to said mammal a therapeutically effective amount of a compound of Formula (I): 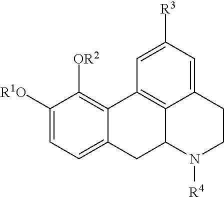
wherein:
R¹, R², and R⁴ are independently chosen from H, substituted or unsubstituted alkyl, substituted or unsubstituted alkenyl, and substituted or unsubstituted alkynyl;
R³ is chosen from H and OR^{S}; and
R⁵ is chosen from H and substituted or unsubstituted alkyl;
or a pharmaceutically acceptable salt form thereof; a therapeutically effective amount of a compound of Formula (II): 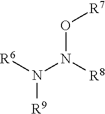
wherein:
R⁶, R⁷, and R⁸ are independently chosen from H and substituted or unsubstituted alkyl; and
R⁹ is chosen from a substituted or unsubstituted cycloalkyl and substituted or unsubstituted heterocycloalkyl;
or a pharmaceutically acceptable salt form thereof; a therapeutically effective amount of a compound of Formula (III): 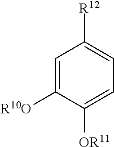
wherein:
R¹⁰ and R¹¹ are independently chosen from H and substituted or unsubstituted alkyl; and
R¹² is chosen from: 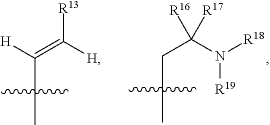
(CH₂)ₙC(O)OR²², and substituted or unsubstituted alkylaryl;
wherein:
R¹³ is chosen from C(O)NR¹⁴R¹⁵, C(O)OR¹⁴, substituted or unsubstituted cycloalkyl, substituted or unsubstituted heterocycloalkyl, substituted or unsubstituted aryl, and substituted or unsubstituted heteroaryl;
R¹⁴ is chosen from H and substituted or unsubstituted alkyl;
R¹⁵ is a substituted or unsubstituted alkylaryl;
R¹⁶ is chosen from H and substituted or unsubstituted alkyl;
R¹⁷ is chosen from H and C(O)OR²⁰;
R¹⁸ and R¹⁹ are chosen from H, C(O)R²¹, and amino;
R²⁰ and R²¹ are independently chosen from H and substituted or unsubstituted alkyl;
R²² is H or a substituted or unsubstituted alky; and
n is an integer from 1 to 6;
wherein if R¹⁶ is methyl, then at least one of R¹⁸ and R¹⁹ is not H;
or a pharmaceutically acceptable salt form thereof; or a therapeutically effective amount of a compound of Formula (IV): 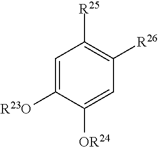
wherein:
R²³ and R²⁴ are independently chosen from H and substituted or unsubstituted alkyl;
R²⁵ is a substituted or unsubstituted heterocycloalkyl and R²⁶ is H, or R²⁵ and R²⁶ come together to form a substituted or unsubstituted cycloalkyl or a substituted or unsubstituted heterocycloalkyl;
or a pharmaceutically acceptable salt form thereof.

2. The method of claim 1, wherein R¹ and R² are H.

3. The method of claim 1, wherein R⁴ is chosen from a substituted or unsubstituted alkyl and a substituted or unsubstituted alkenyl.

4.-5. (canceled)

6. The method of claim 1, wherein R³ is OR⁵.

7. (canceled)

8. The method of claim 1, wherein the compound of Formula (I) is chosen from: 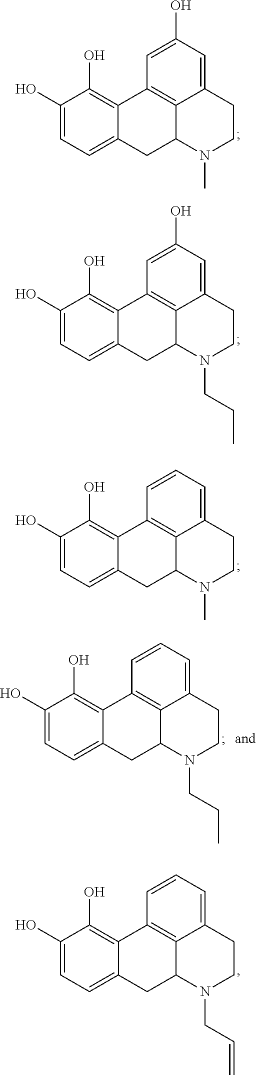
or a pharmaceutically acceptable salt form thereof.

9. The method of claim 1, wherein the compound of Formula (I) is chosen from: 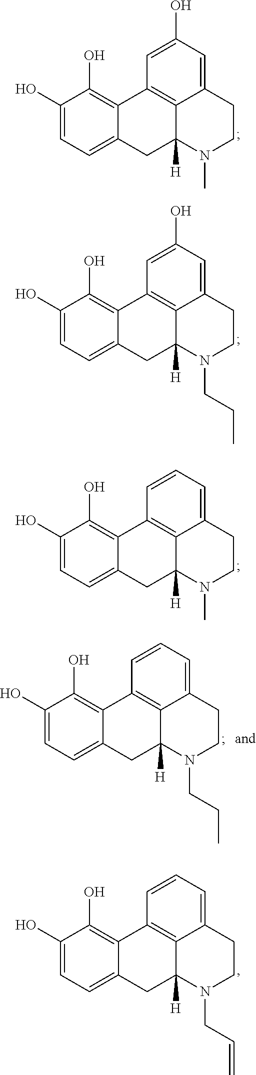
or a pharmaceutically acceptable salt form thereof.

10. (canceled)

11. The method of claim 1, wherein R⁶ is a substituted or unsubstituted alkyl.

12.-13. (canceled)

14. The method of claim 1, wherein R⁹ is a substituted or unsubstituted cycloalkyl.

15. The method of claim 14, wherein R⁹ is chosen from 2,5-benzoquinonyl and 3,4-benzoquinonyl.

16. The method of claim 1, wherein the compound of Formula (II) is chosen from: 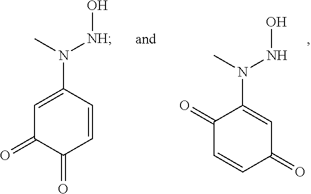
or a pharmaceutically acceptable salt form thereof.

17.-18. (canceled)

19. The method of claim 1, wherein R¹² is

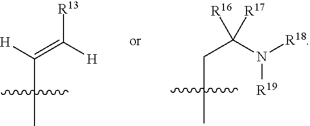

20. The method of claim 19, wherein R¹³ is C(O)OH.

21. The method of claim 19, wherein R¹³ is chosen from a substituted or unsubstituted heterocycloalkyl and substituted or unsubstituted aryl.

22.-23. (canceled)

24. The method of claim 19, wherein R¹⁷ is COOH.

25. (canceled)

26. The method of claim 1, wherein the compound of Formula (III) is chosen from: 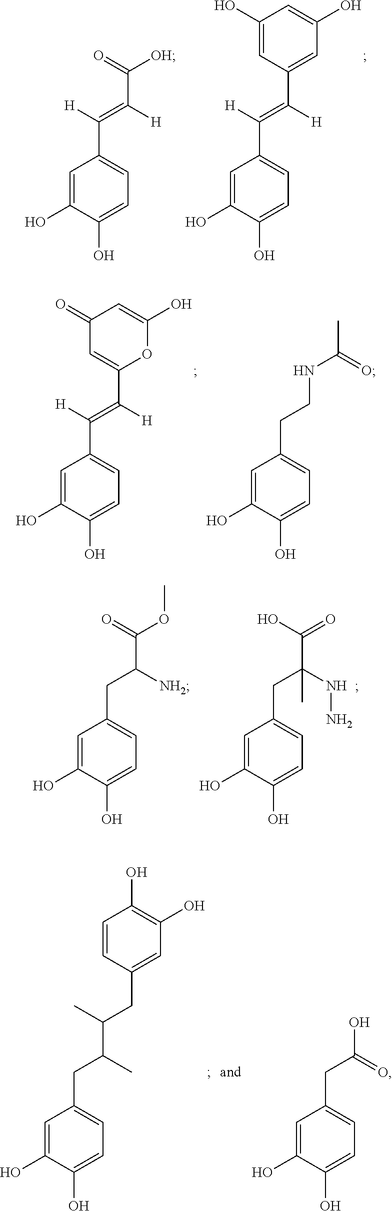
or a pharmaceutically acceptable salt form thereof.

27. The method of claim 1, wherein the compound of Formula (III) is chosen: 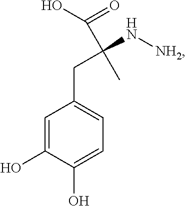
or a pharmaceutically acceptable salt form thereof.

28. (canceled)

29. The method of claim 1, wherein the compound of Formula (IV) is chosen from: 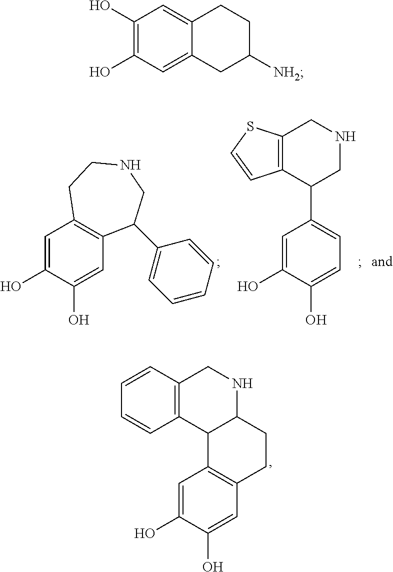
or a pharmaceutically acceptable salt form thereof.

30. The method of claim 1, wherein the compound of Formula (IV) is: 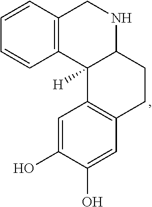
or a pharmaceutically acceptable salt form thereof.

31. A method for inhibiting APOBEC3G activity in a mammal, said method comprising administering to said mammal a therapeutically effective amount of a compound chosen from: 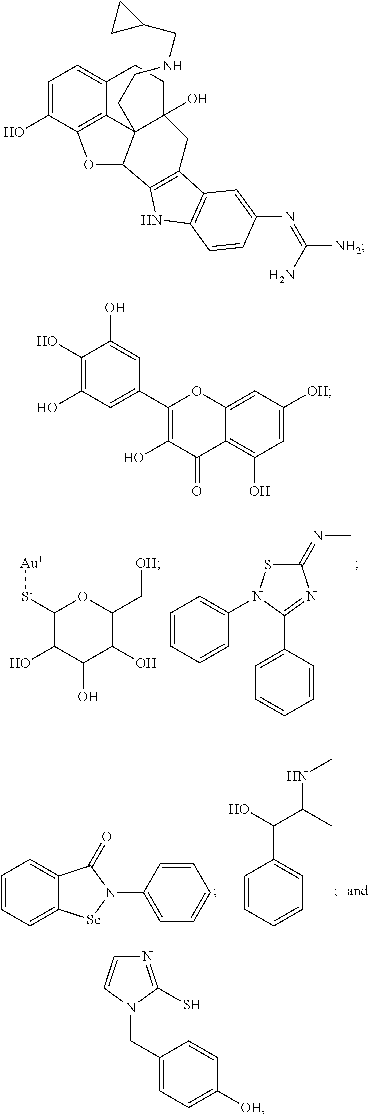
or a pharmaceutically acceptable salt form thereof.

32. A method for inhibiting APOBEC3G activity in a mammal, said method comprising administering to said mammal a therapeutically effective amount of a compound chosen from: 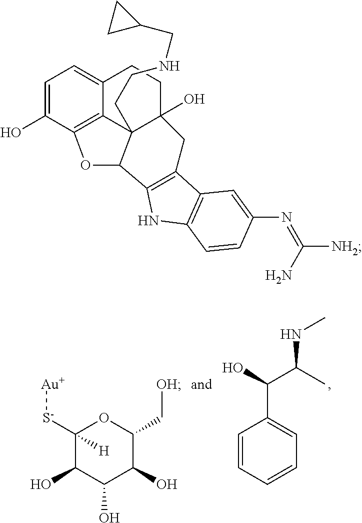
or a pharmaceutically acceptable salt form thereof.

33. The method of claim 1, wherein said mammal is a human patient.

34. The method of claim 1, wherein said mammal is a mouse, rat, non-human primate, or artiodactyl.

35. A method of identifying inhibitors of HIV-1 mutation, said method comprising measuring HIV-1 mutation rate in the presence and absence of an inhibitor of the anti-retroviral activity of APOBEC3G

36. A method of identifying inhibitors of the anti-retroviral activity of APOBEC3G, said method comprising
a) assaying cytosine deaminase activity of APOBEC3G. in the presence and absence of a compound, wherein said compound is identified as an inhibitor of cytosine deaminase activity if cytosine deaminase activity is reduced in the presence of said compound; and
b) assaying replication of Vif-deficient HIV-1 in an APOBEC3G-expressing human T cell line in the presence of said inhibitor of cytosine deaminase activity, wherein said compound is identified as an inhibitor of the anti-retroviral activity ofAPOBEC3G if Vif-deficient HIV-1 replicates in said APOBEC3G-expressing human T cell line.

37. The method of claim 36, said method further comprising comparing time for Vif-proficient HIV-1 to develop drug resistance in the presence and absence of said inhibitor of cytosine deaminase activity, wherein said compound is identified as an inhibitor of the anti-retroviral activity ofAPOBEC3G when time to develop resistance is increased in the presence of said compound relative to the absence of said compound.

38. The method of claim 36, wherein said APOBEC3G-expressing human T cell line is CEM-SS or CEM-SS-A3G.

39. The method of claim 37, further comprising assessing mutation profile of drug resistant Vif-proficient HIV-1.

40. The method of claim 36, wherein cytosine deaminase activity is assayed by
i) incubating a source of APOBEC3G, a single-strand DNA oligonucleotide substrate, and uracil DNA glycosylase in the presence and absence of said compound, wherein said oligonucleotide substrate comprises a fluorescent donor molecule on its 5′ end and a fluorescent acceptor molecule on its 3′ end; and
ii) measuring fluorescence, wherein a decrease in fluorescence in the presence of said compound relative to the fluorescence in the absence of said compound indicates said compound is an inhibitor of cytosine deaminase activity.

41. The method of claim 40, wherein said source of APOBEC3G is a cell lysate or a purified APOBEC3G protein.

42. (canceled)
